(19) United States
(12) Patent Application Publication
(10) Pub. No.: US 2024/0382487 A1
(43) Pub. Date: Nov. 21, 2024

(54) NOVEL COPI/ARF1-LIPOLYSIS PATHWAY INHIBITOR AND COMPOUND FOR ERADICATING CANCER STEM CELLS AND INDUCING DAMP-MEDIATED ANTI-TUMOR IMMUNE RESPONSE

(71) Applicants: FUDAN UNIVERSITY, Shanghai (CN); GREATER BAY AREA INSTITUTE OF PRECISION MEDICINE (GUANGZHOU), GUANGZHOU (CN)

(72) Inventors: Xianyu HOU, Shanghai (CN); Yuetong WANG, Shanghai (CN); Na WANG, Shanghai (CN)

(21) Appl. No.: 18/026,034

(22) PCT Filed: Aug. 3, 2021
(86) PCT No.: PCT/CN2021/110373

§ 371 Date: Mar. 13, 2023

Related U.S. Application Data
(60) Provisional application No. 63/060,899, filed on Aug. 4, 2020.

(51) Int. Cl. A61K 31/519; A61P 35/00; C07D 495/04

(52) U.S. Cl. CPC A61K 31/519; A61P 35/00; C07D 495/04

(57) ABSTRACT

The present invention relates to use of a novel Arf1 inhibitor; a method for treating refractory, recurrent or metastatic cancer using these compounds; an investigate method for selectively killing cancer stem cells and cancer cells using the compounds and a specific dosing regimen; an investigate method for targeting tumor stem cells and inducing an anti-tumor immune response by inhibiting an Arf1 pathway, particularly a COPI/Arf1-lipolysis-β-oxidation pathway; a method for treating Arf1 pathway activity related diseases using a new compound in mammals; a process for preparing the compounds and intermediates thereof; and a pharmaceutical composition of a related compound.

### TECHNICAL FIELD

The invention generally relates to inhibitors of the COPI/Arf1 mediated lipolysis-β-oxidation pathway in the treatment of diseases. More specifically, the invention relates to the usage of inhibitors of the COPI/Arf1 mediated lipolysis-β-oxidation pathway in targeting cancer stem cells, activating immune response and treating diseases. More specifically, the present invention relates to 1H-adduct-5-formaldehyde 6,7,8,9-tetrahydro-5H-cycloheptyl [4,5] thiopheno [2,3-d]kazaka-4-base gland, 1H-adduct-5-formaldehyde 5,6,7,8,9,10-hexahydrocyclooctyl [4,5]thiopheno [2,3-5] thiopheno [2,3-5] 4-base gland, and related compounds that inhibit Arf1, target cancer stem cells and activate anti-tumor immune response, and are used to treat malignant diseases. The invention also relates to the treatment of refractory, recurrent or metastatic cancer, the preparation method of related compounds and intermediates, and the pharmaceutical composition of related compounds.

### BACKGROUND

In the United States alone, there are hundreds of thousands of cancer deaths every year. Although progress has been made in the treatment of some types of cancer through surgery, radiotherapy and chemotherapy, many types of cancer are basically incurable. Even if there is an effective treatment for specific cancer, the side effects of this treatment may be serious and lead to a significant decline in the quality of life.

Immunotherapies, especially checkpoint blockade therapies, are beginning to fundamentally change the paradigm of treating cancer patients (Sharma et al., 2017). However, despite recent clinical success, immunotherapy has unfortunately only provided help to a small subset of cancer patients. Most patients derive little benefit, as most emerging tumors have developed immune evasion mechanisms in the tumor microenvironment, including dysfunctional T cells and lack of T cell infiltration (jerby arnon et al., 2018; Sharma et al., 2017). But pathogen (bacterial or viral)—induced immune responses are mostly intact in cancer patients. These pathogens express unique molecules called pathogen associated molecular patterns (PAMPs) that are recognized by pattern recognition receptors (PRRS) of the innate immune system to activate anti-infective immune responses. This induced immune response can be used to regress tumors, as first demonstrated by William Coley (balkwill, 2009). In the 1890s, he succeeded in regressing a number of sarcomas and/or lymphomas after in vivo injection of streptococcal cultures in patients. Some of the injected bacteria may trigger PAMP induced antitumor immune responses.

It was recently discovered that the immune system can be induced not only by PAMPs but also by endogenous signals known as danger/damage associated molecular patterns (damps) that are released from damaged or stressed cells in the absence of microbial components (jerby amon et al., 2018; Sharma et al., 2017). The DAMPS are analogues of PAMPs, molecules present in specific cellular compartments and not or only limited expression under physiological conditions. But it is strongly induced, and then under conditions of stress or injury damps translocate to the cell surface or extracellular space. The most important damps include: (I) pre apoptotic exposure of the endoplasmic reticulum (ER)—anchored molecular chaperone calreticulin (Calr) on the cell surface; (II) the release of non-histone nuclear protein high mobility group box 1 (HMGB1) into the extracellular space; (III) active secretion of ATP. Surface exposed Calr will facilitate engulfment of tumor associated antigens by binding to LRP1/CD91 on dendritic cells (DCS), and extracellular HMGB1 will bind to DCS Toll like receptor 4 (TLR4) and activates the TLR4-MyD88 pathway to support DC maturation and prevent accelerated destruction of lysosomes engulfing metabolized foreign material for antigen processing and presentation to cytotoxic T lymphocytes (CTLs). In addition, dying tumor cells secrete ATP that activates IFN-y secreting T cells via the p2rx7 receptor, which in turn mediates antitumor immune responses (ghiringhelli et al., 2009). Taken together, these DAMPs differ in signaling pathways that promote the development of host responses to tumor specific adaptive immunity. These can inhibit or even completely eradicate drug-resistant tumor cells. However, the intrinsic molecular mechanisms that trigger damp release from tumor cells and induce antitumor immune responses in vivo are not well understood.

Cancer stem cells (CSCs) are a subset of cell populations in the tumor that are in a stem cell state and possess stem cell characteristics. CSCs may contribute to therapy resistance, tumor metastasis, disease recurrence, and ultimately patient death (Batlle and Clevers, 2017; Lytle et al., 2018; shibue and Weinberg, 2017). The ultimate goal of CSC research is to identify pathways that selectively regulate CSC survival and then target this pathway to eradicate CSCs some CSCs are generated from the transformation of normal stem cells and others by reprogramming non-CSC cancer cells to a stem cell state. CSCs and normal stem cells share many properties. Thus, pathways regulating normal or transformed stem cells may also regulate CSC. We previously found that the COPI/Arf1 mediated lipolysis pathway selectively maintains stemness and transformed stem cells in Drosophila and that knockdown of this pathway causes stem cell necrosis (Singh et al., 2016). In a recent study, we showed that knockdown of Arf1 in mice disrupts lipid metabolism and leads to accumulation of lipid droplets and causing metabolic stress induced by mitochondrial defect and endoplasmic reticulum stress. Metabolic stress selectively kills progenitor cells, stem cells, and CSCs via necrosis in mice dying CSCs release damps to activate DCS, which further enhance T cell infiltration and activation, and then stimulate the body's antitumor immune response. Our data indicate that knockdown of the Arf1 pathway not only kills CSCs but also elicits tumor specific immune responses and converts dying CSCs into therapeutic vaccines, which actually improves the long-term efficacy of treatment (Wang et al., 2020).

Our findings that Arf1 inhibition stimulates T cell infiltration and activation may provide the basis for novel therapeutic strategies that exploit DAMPs-mediated induced anti-tumor immunity in cancer patients. This may be another promising direction of cancer immunotherapies in addition to the successful checkpoint blockades. The new therapies may include approaches that reduce Arf1 activity as well as block Arf1-mediated lipolysis pathway and associated β-oxidation. We tested 10 reported Arf1 inhibitors (Bill et al., 2011; Boal et al., 2010; Feng et al., 2004; Newton et al., 2006; Ohashi et al., 2012; Sáenz et al., 2009; Sorieul et al., 2011; Viaud et al., 2007), including brefeldin A (BFA), Golgicide A (GCA), LM11, Secin16, Secin H3, Secin B7, Exo1, Exo2, LG8, and LG18, on Drosophila stem cell tumors and normal human bone marrow hematopoietic stem cells (HSCs). We found that these known inhibitors are either not very effective in killing Drosophila stem cell tumors or too toxic (killing normal HSCs). We also synthesized and tested new inhibitors based on Exo2 and found that Du 101 and Du 102 (described below) are effective and have relatively low toxicity.

### SUMMARY

The present invention is based on our recent finding that ablation of the COPI/ARF1-lipolysis-β-oxidation pathway eradicates cancer stem cells and induces DAMP-mediated Anti-tumor Immune Responses. Accordingly, a first aspect of the invention is directed to a method of eliminating tumors where the method comprises inhibiting at least some, most, or substantially all (e.g., 30%, 40%, 50%, 60%, 70%, 80%, 90%, or 95%), of the Arf1 pathway activity in the cancer patients through a Arf1 pathway inhibitor.

In one embodiment, the Arf1 pathway inhibitor is isolated, purified or synthetic, and can be selected from the group consisting of a small molecule Arf1 inhibitor, an RNAi agent against Arf1, an antisense agent against Arf1, a peptidomimetic Arf1 inhibitor, and a G-quartet oligodeoxynucleotides Arf1 inhibitor. The mechanism of inhibition can be selected from the group consisting of substantially inhibiting Arf1 GTPase activity, substantially inhibiting Arf1-pathway-regulated lipolysis activity, substantially inhibiting Arf1-pathway-regulated β-oxidation activity, and substantially inducing cell necrosis through inhibiting Arf1 pathway.

In one embodiment, the inhibitor is a compound selected from the group consisting of compounds 102, 104-111 described above, especially Du 101 and Du 102, an enantiomer, diastereomer, tautomer, and a salt or solvate thereof (hereafter referred to as the “Compound of the Invention”).

The present invention provides method of inhibiting or reducing growth of a tumor or cancer comprising contacting the tumor with an effective amount of Compound of the Invention. The compound inhibits growth of the tumor or cancer or the compound reduces the size of the tumor or cancer. The compound increases expression of MHC-I and MHC-II. The compound increases infiltration and activation of T cells into the tumor when compared to DMSO. The compound increases expression of T cell activation markers, such as GzmA, GzmB and Perforin. In another embodiment, the compound increases the expression of at least one inflammatory cytokine or chemokine, which can be selected from the group consisting of IFNγ, IL-1β, Ccl5, Cxc10, Cxc11, an Ccl22. In certain embodiments, the compound is co-administered with at least one anti-PD-1 antibody, the compound and PD-1 blockage had a synergistic effect. In certain embodiments, vaccination with the compound treated cancer cells protects animals from developing tumors. In certain embodiments, treatment with the compound has effects of “one stone killing two birds”, not only kills CSCs but also elicits a tumor-specific immune response and converts dying CSCs into a therapeutic vaccine, which results in the long-term efficacy of the treatment.

In a second aspect, the present invention provides a method of treating or preventing a disorder associated with Arf1 pathway activity in a subject, the method comprising administering to the subject a therapeutically effective amount of a pharmaceutical composition comprising the Compound of the Invention such that Arf1 pathway activity is reduced. In one feature, Arf1 pathway activity can be identified by testing Arf1 GTPase activity or a surrogate upstream or downstream regulator of Arf1 GTPase activity. The disorder can be a cancer. In one embodiment, the cancer is known to have Arf1 pathway activities, and include but are not limited to: breast cancer, head and neck cancer, lung cancer, ovarian cancer, pancreatic cancer, colorectal carcinoma, prostate cancer, renal cell carcinoma, melanoma, hepatocellular carcinomas, cervical cancer, sarcomas, brain tumors, gastric cancers, multiple myeloma, leukemia, and lymphomas. The disorder may also be a non-cancerous condition known to be associated with Arf1 pathway activity, and in one embodiment, is selected from the group consisting of an autoimmune disease, an inflammatory disease, inflammatory bowel diseases, arthritis, autoimmune demyelination disorder, Alzheimer's disease, stroke, ischemia reperfusion injury, multiple sclerosis, and other inflammatory or neurodegenerative diseases.

In a second aspect, the present invention provides a method of treating or preventing a disorder associated with Arf1 pathway activity in a subject, the method comprising administering to the subject a therapeutically effective amount of a pharmaceutical composition comprising the Compound of the Invention such that Arf1 pathway activity is reduced. In one feature, Arf1 pathway activity can be identified by testing Arf1 GTPase activity or a surrogate upstream or downstream regulator of Arf1 GTPase activity. The disorder can be a cancer. In one embodiment, the cancer is known to have Arf1 pathway activities, and include but are not limited to: breast cancer, head and neck cancer, lung cancer, ovarian cancer, pancreatic cancer, colorectal carcinoma, prostate cancer, renal cell carcinoma, melanoma, hepatocellular carcinomas, cervical cancer, sarcomas, brain tumors, gastric cancers, multiple myeloma, leukemia, and lymphomas. The disorder may also be a non-cancerous condition known to be associated with Arf1 pathway activity, and in one embodiment, is selected from the group consisting of an autoimmune disease, an inflammatory disease, inflammatory bowel diseases, arthritis, autoimmune demyelination disorder, Alzheimer's disease, stroke, ischemia reperfusion injury, multiple sclerosis, and other inflammatory or neurodegenerative diseases.

In a third aspect, the present invention provides a method of inhibiting cellular Arf1 pathway activity in a cell. The method includes administering to the cell an effective amount of the Compound of the Invention such that Arf1 pathway activity in the cell is reduced, for example, by at least 20%, 30%, 40%, 50%, 60%, 70%, 80%, 90% or 95%. In one embodiment, the cell is a CSC, or otherwise cancerous. The method may induce cell death. The method can be carried out in vitro or in vivo.

In a fourth aspect, the present invention provides a pharmaceutical composition that comprises the Compound of the Invention, i.e., a compound selected from the group consisting of compounds 102, 104-111 described above, and a pharmaceutically acceptable salt or solvate thereof, and a pharmaceutically-acceptable excipient, carrier, or diluent. In one feature, the composition is suitable for oral, nasal, topical, rectal, vaginal or parenteral administration, or intravenous, subcutaneous or intramuscular injection.

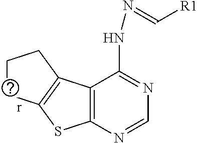

In a fifth aspect, the present invention also provides a process of preparing some of the Compounds of the Invention. The method prepares a compound of formula I,
Where r=1-6; R1 is selected from the group consisting of one or more substituents or unsubstituted phenyl,

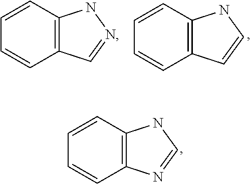

substituents selected from the group consisting of halo, C₁₋₆ alkyl, haloalkyl, OH, OCH3, O(CH₂)nCH₃ Cyclopropyloxy group, OC(CH₃)₃, OCH(CH₃)₂, NH₂, NO₂,
N(CH₃)₂, NH(CH₂)nCH₃, CN, N₃, etc., which the median N was 1-9. Specifically, wherein the inhibitors targeting the Arf1 pathway are compounds as follows:

TABLE 1

| | |
|---|---|
| 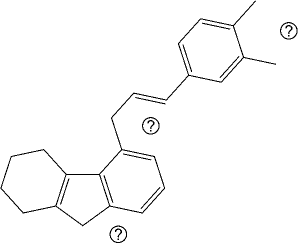 [SMILES: Cc1ccc(/C=C/Cc2cccc3c2C2=C(CCCC2)C3)cc1C] | 102 |
| 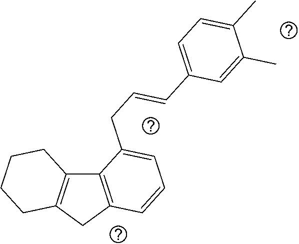 [SMILES: Cc1ccc(/C=C/Cc2cccc3c2C2=C(CCCC2)C3)cc1C] | 104 |
| 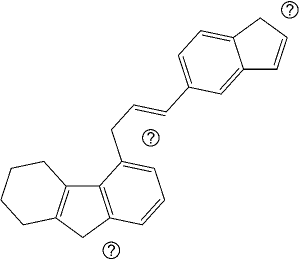 [SMILES: C1=Cc2cc(/C=C/Cc3cccc4c3C3=C(CCCC3)C4)ccc2C1] | 105 |
| 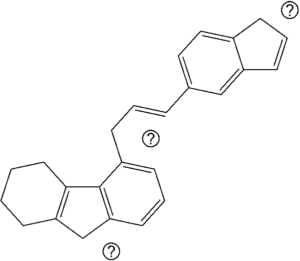 [SMILES: C1=Cc2cc(/C=C/Cc3cccc4c3C3=C(CCCC3)C4)ccc2C1] | 106 |
| 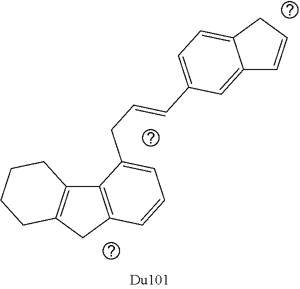 [SMILES: C1=Cc2cc(/C=C/Cc3cccc4c3C3=C(CCCC3)C4)ccc2C1] | 107 |
| 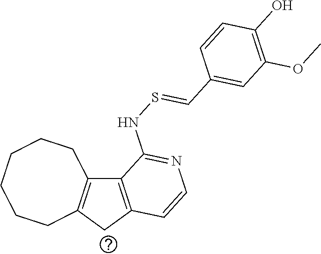 | 108 |
| 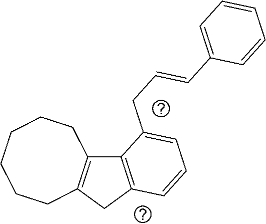 [SMILES: C(=C\c1ccccc1)/Cc1cccc2c1C1=C(CCCCCC1)C2] | 109 |
| 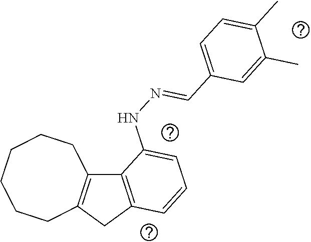 [SMILES: Cc1ccc(/C=N/Nc2cccc3c2C2=C(CCCCCC2)C3)cc1C] | 110 |
| 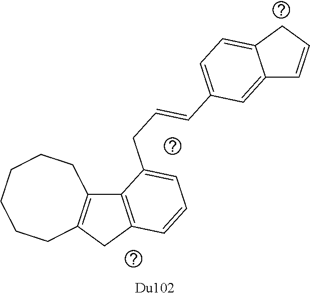 [SMILES: C1=Cc2cc(/C=C/Cc3cccc4c3C3=C(CCCCCC3)C4)ccc2C1] | 111 |

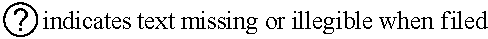

In a sixth aspect, the present invention provides a process of preparing the compound 1H-indole-5-carbaldehyde6,7,8,9-tetrahydro-5H-cyclohepta [4.5] thieno[2,3-d] pyrimidin-4-ylhydrazone.

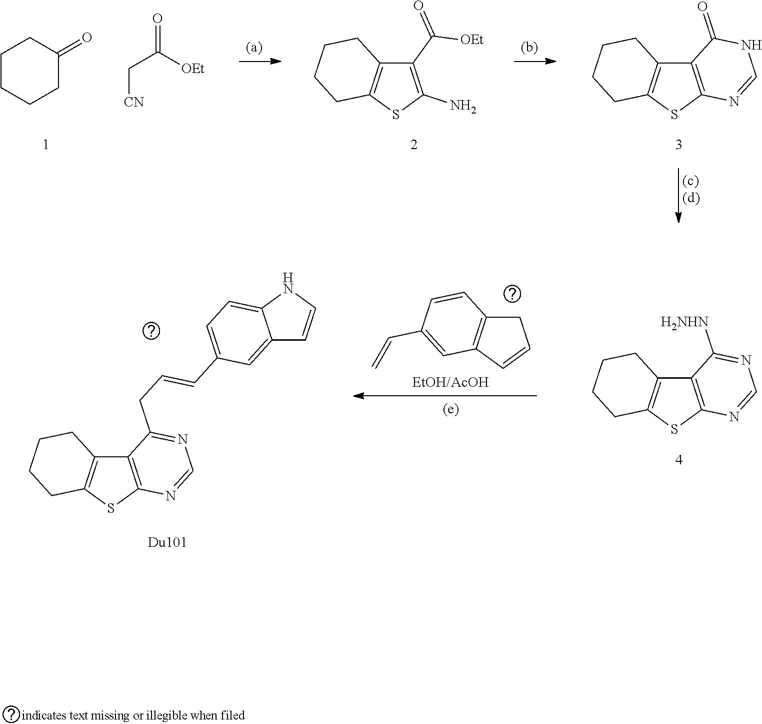

In a seventh aspect the present invention provides a process of preparing the compound 1H-indolo-5-carbaldehyde 5,6,7,8,9,10-hexahydrocycloocta[4,5]thieno[2,3-d]pyrimidin-4-ylhydrazone or 111 or DU 102 described above.

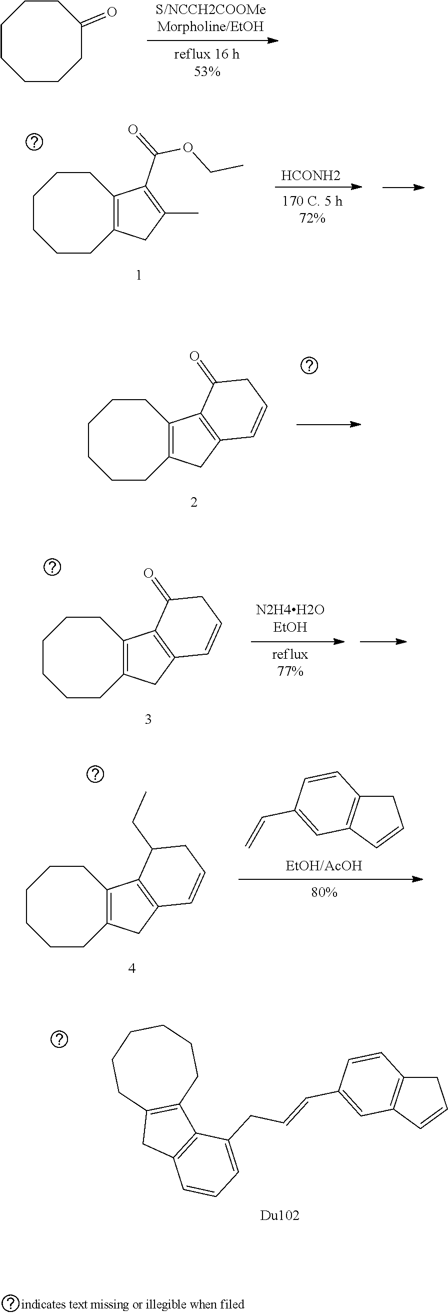

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without parting from the spirit and scope of the invention. All references cited herein are incorporated by reference as if each had been individually incorporated.

### I. Definitions

As used herein, the singular form “a”, “an”, and “the” include plural references unless the context clearly dictate otherwise. For example, the term “a cell” includes a plurality of cells including mixtures thereof.

As used herein, the terms “cancer stem cell(s)” and “CSC(s)” are interchangeable. CSCs are mammalian, and in preferred embodiments, these CSCs are of human origin, but they are not intended to be limited thereto. Cancer stem cells are defined and functionally characterized as a population of cells originating from a tumor that: (1) have extensive proliferative capacity; 2) are capable of asymmetric cell division to generate one or more kinds of differentiated progeny with reduced proliferative or developmental potential; and (3) are capable of symmetric cell divisions for self-renewal or self-maintenance. Other common approaches to characterize CSCs involve morphology and examination of cell surface markers, transcriptional profile, and drug response. CSCs are also called in the research literature tumor/cancer initiating cells, cancer stem-like cells, stem-like cancer cells, highly tumorigenic cells, tumor stem cells, solid tumor stem cells, drug survival cells (DSC), drug resistant cells (DRCs) or super malignant cells.

As used herein, the terms “cancer” and “cancerous” refer to or describe the physiological condition in mammals in which a population of cells are characterized by unregulated cell growth. “Cancer cells” and “tumor cells” as used herein refer to the total population of cells derived from a tumor including both non-tumorigenic cells, which comprise the bulk of the tumor cell population, and tumorigenic stem cells (cancer stem cells). Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

“Tumor” as used herein refers to any mass of tissue that result from excessive cell growth or proliferation, either benign (noncancerous) or malignant (cancerous) including precancerous lesions.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Terms such as “treating” or “treatment” or “to treat” or “alleviating” or “to alleviate” as used herein refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those already with the disorder; those prone to have the disorder; and those in whom the disorder is to be prevented. A subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

As used herein, the term “inhibiting”. “to inhibit” and their grammatical equivalents, when used in the context of a bioactivity, refer to a down-regulation of the bioactivity, which may reduce or eliminate the targeted function, such as the production of a protein or the phosphorylation of a molecule. In particular embodiments, inhibition may refer to a reduction of about 20%, 30%, 40%, 50%, 60%, 70%, 80%, 90% or 95% of the targeted activity. When used in the context of a disorder or disease, the terms refer to success at preventing the onset of symptoms, alleviating symptoms, or eliminating the disease, condition or disorder.

The term “lipolysis” as used therein means the breakdown of lipids and involves hydrolysis of triglycerides into glycerol and free fatty acids. Predominantly occurring in adipose tissue, lipolysis is used to mobilize stored energy during fasting or exercise.

The term “GTPase” as used therein means hydrolase enzymes that can bind and hydrolyze guanosine triphosphate (GTP). The GTP binding and hydrolysis takes place in the highly conserved G domain common to all GTPases.

The term “β-oxidation” as used therein means the catabolic process by which fatty acid molecules are broken down in the cytosol in prokaryotes and in the mitochondria in eukaryotes to generate acetyl-CoA, which enters the citric acid cycle, and NADH and FADH₂, which are co-enzymes used in the electron transport chain. It is named as such because the beta carbon of the fatty acid undergoes oxidation to a carbonyl group. Beta-oxidation is primarily facilitated by the mitochondrial trifunctional protein, an enzyme complex associated with the inner mitochondrial membrane, although some fatty acids are oxidized in peroxisomes.

The term “necrotic cell death” as used therein means when cells are exposed to extreme variance from physiological conditions (e.g., hypothermia, hypoxia) which may result in damage to the plasma membrane. Under physiological conditions direct damage to the plasma membrane is evoked by agents like complement and lytic viruses. Necrosis begins with an impairment of the cell's ability to maintain homeostasis, leading to an influx of water and extracellular ions. Intracellular organelles, most notably the mitochondria, and the entire cell swell and rupture (cell lysis). Due to the ultimate breakdown of the plasma membrane, the cytoplasmic contents including lysosomal enzymes are released into the extracellular fluid. Therefore, in vivo, necrotic cell death is often associated with extensive tissue damage resulting in an intense inflammatory response.

“Immune condition” or “immune disorder” encompasses. e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist irradiation by the immune system. “Cancerous condition” includes. e.g., cancer, cancer cells, tumors, angiogenesis, and precancerous conditions such as dysplasia.

The term “cytotoxic T cell” as used therein means a T lymphocyte (a type of white blood cell) that kills cancer cells, cells that are infected (particularly with viruses), or cells that are damaged in other ways. Most cytotoxic T cells express T-cell receptors (TCRs) that can recognize a specific antigen. An antigen is a molecule capable of stimulating an immune response, and is often produced by cancer cells or viruses. Antigens inside a cell are bound to class I MHC molecules, and brought to the surface of the cell by the class I MHC molecule, where they can be recognized by the T cell. If the TCR is specific for that antigen, it binds to the complex of the class I MHC molecule and the antigen, and the T cell destroys the cell.

In order for the TCR to bind to the class I MHC molecule, the former must be accompanied by a glycoprotein called CD8, which binds to the constant portion of the class I MHC molecule. Therefore, these T cells are called CD8+ T cells.

Once cytotoxic CD8⁺ T cells (CTLs) recognize their target cells in the periphery, the CTLs are activated and a highly specialized cell-to-cell contact structure called the immune synapse (IS) formed between the T cell and its target cell. Activation of the TCR results in the engagement of Src family kinases, Lek and Fyn, and the recruitment of the Syk family kinase ZAP-70 to the TCR, where these tyrosine kinases become activated. Activated ZAP70 in turn phosphorylates the adaptor protein linker for activation of T cells (LAT) leading to the formation of the LAT signalosome, which includes phospholipase Cγ1 (PLCγ1) and SLP76 (SH2 domain-containing leukocyte protein of 76 kDa). PLCγ1 converts phosphatidylinositol-4,5-bisphosphate (PIP₂) into DAG (diacylglycerol) and IP3 (inositol-1,4,5-trisphosphate). DAG accumulates at the immune synapse, which leads to the recruitment of novel protein kinase Cs (PKCs), including PKCθ. PKCθ then facilitates centrosome (microtubule organizing center or MTOC) polarization by reciprocally localizing dynein to the synapse, which pulls the MTOC in the direction of the synapse, and non-muscle myosin II (NMII) to the opposite side of the cell, where it pushes the MTOC toward the synapse. The docking of the MTOC beneath the IS ensures the dynein- and Ca²⁺-dependent targeted delivery and exocytosis of the granule content directly into the synaptic cleft, which causes target cell lysis by the concerted action of granzymes and perforin (reviewed in: de la Roche et al., 2016).

The term “pharmaceutically-acceptable excipient, carrier, or diluent” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

The compounds of the present invention may form salts which are also within the scope of this invention. Reference to a compound of the present invention herein is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., nontoxic, physiologically acceptable) salts are preferred, although other salts are also useful, e.g., in isolation or purification steps which may be employed during preparation. Salts of the compounds of the present invention may be formed, for example, by reacting a compound 102 or 104-111 with an amount of acid or base, such as an equivalent amount, in a medium such as one in which the salt precipitates or in an aqueous medium followed by lyophilization.

Solvates of the compounds of the invention are also contemplated herein. Solvates of the compounds of the present invention include, for example, hydrates.

The present invention provides methods of treating proliferative disorders. e.g., cancer, tumors, etc., with a compound selected from the group consisting of compounds 102, 104-111 described above, especially Du 101 and Du 102. Recently, we have identified a novel connection that links lipid metabolism, CSC death and anti-tumor immunity. CSCs may be responsible for treatment resistance and immune evasion. CSC signals can modulate lymphocyte infiltration into tumor and alter the tumor microenvironment (Lytle et al., 2018). We have demonstrated that ablating Arf1-mediated lipid metabolism in CSCs resulted in metabolic stress and subsequent cellular responses including releasing DAMPs, which promotes anti-tumor immunity through activating DCs, enhancing T cell infiltration and activation (manuscript in submission). We further demonstrated that Arf11 ablation and PD-1 blockage had a synergistic effect. Consistently, TCGA data analysis shows an inverse correlation between Arf1 expression and T cell infiltration and activation as well as better survival probability in various human cancers. Our results reveal that knockdown of the Arf1 pathway has effects of “one stone killing two birds”, not only kills CSCs but also elicits a tumor-specific immune response and converts dying CSCs into a therapeutic vaccine, which results in the long-term efficacy of the treatment (manuscript in submission).

The present invention provides evidence that the new compounds inhibit Arf1 pathway activity and induce anti-tumor immune responses through increasing the expression of chemokines and inflammatory cytokines as well as enhancing T cell infiltration and activation. We further demonstrated that treatment with the compound and PD-1 blockage had a synergistic effect. Treatment with the compound should therefore provide a significant improvement for tumor treatment.

The data provided herein, combined with recent breakthroughs in anti-tumor immunity research, allows the present invention to provide an array of methods directed at inhibiting CSCs, or treating cancers that have CSCs in specific or cancers in general. Also provided herein are methods directed at inhibiting Arf1 pathway activity in cells, or treating disorders, both cancerous and non-cancerous, that are associated with Arf1 pathway activities. The present invention also provides related methods (e.g., manufacturing and drug candidate screening), materials, compositions and kits.

This method can be used to treat a subject's cancer. Cancers that are known to have CSCs and Arf1-lipolysis-β-oxidation pathway activities are good candidates for such treatment, and include but are not limited to: breast cancer, head and neck cancer, lung cancer, ovarian cancer, pancreatic cancer, colorectal carcinoma, prostate cancer, renal cell carcinoma, melanoma, hepatocellular carcinomas, cervical cancer, sarcomas, brain tumors, gastric cancers, multiple myeloma, leukemia, and lymphomas. In an embodiment, the method is used to treat liver cancers, head and neck cancers, pancreatic cancers, and/or gastric cancers. In another embodiment, the method is used to treat multiple myeloma, brain tumors, and sarcomas.

Further, as CSCs have been demonstrated to be fundamentally responsible for tumorigenesis, cancer metastasis and cancer reoccurrence, any methods of the invention directed to inhibiting CSCs can be practiced to treat cancer that is metastatic, refractory to a chemotherapy or radiotherapy, or has relapsed in the subject after an initial treatment.

In one embodiment, the inhibitor is isolated, purified or synthetic, and can be selected from the group consisting of a small molecule Arf1 inhibitor, an RNAi agent against Arf1, an antisense agent against Arf1, a peptidomimetic Arf1 inhibitor, and a G-quartet oligodeoxynucleotides Arf1 inhibitor. The inhibitor may be isolated or purified from a natural product as well.

The mechanism of inhibition can be selected to target any step in the Arf1 pathway. For example, the inhibitor can substantially inhibit Arf1 GTPase activity, lipolysis reporter, lipid droplet formation, autophagy, and a surrogate upstream or downstream regulator of Arf1 activity or functions.

In one embodiment, the Arf1 inhibitor according to the present invention is: compound 102, 104-111 (Table 1), an enantiomer, diastereomer, tautomer, and a salt or solvate thereof (the “Compound of the Invention”). The present invention also provides both in vitro and in vivo data that the Compound of the Invention reduce tumors.

The Compound of the Invention is not only shown to cause death in abroad spectrum of cancer cells, but also to exhibit selectivity in its cytotoxicity which is critical for developing low-toxicity therapeutics. Selective cytotoxicity as used herein refers to a compound's ability to kill cancer cells while substantially sparing normal cells, sometimes under certain conditions. Normal cells usually refer to healthy, non-tumorigenic cells. Conditions that result in selective cytotoxicity for a drug candidate are hard to predict because they require knowledge of the underlying mechanism of cytotoxicity. For example, to lower the toxicity of an anti-cancer drug that targets microtubule formation during mitosis presents quite different factors to work with than a drug that blocks cellular metabolic processes. A suitable condition for engendering selective cytotoxicity needs to balance the need for the drug to be toxic enough to effectively kill cancer cells while tolerable enough to normal cells. For instance, if lower concentration is used, that often means prolonged infusion is needed to kill cancer cells.

From data generated in the examples of this invention, it appears that selective cytotoxicity can be achieved for the Compound of the Invention if affected cells are not exposed to a critical concentration of the compound continuously beyond a certain duration. In a method aimed at selectively killing cancer cells in a subject, a pharmaceutical composition that has the Compound of the Invention is administered to the subject such that the compound concentration in the subject's plasma is not maintained above a critical concentration for more than 24 hours after each dose. This method can be used to treat all cancers, including any of the groups of cancers described here, and to treat Arf1-associated disorder, an exemplary list of which is already provided above and is not repeated here. Alternatively, the duration can be further restricted to 12, 16, and 20 hours after each dose. The critical concentration for each compound may vary. In various embodiments of the present invention, the critical concentration is about 100 μM, about 50 μM, about 20 μM, or about 10 μM.

### Uses

The present invention provides methods of treating a proliferative condition or disorder, e.g., cancer of the uterus, cervix, breast, prostate, testes, penis, gastrointestinal tract, e.g., esophagus, oropharynx, stomach, small or large intestines, colon, or rectum, kidney, renal cell, bladder, bone, bone marrow, skin, head or neck, skin, liver, gall bladder, heart, lung, pancreas, salivary gland, adrenal gland, thyroid, brain, e.g. gliomas, ganglia, central nervous system (CNS) and peripheral nervous system (PNS), and immune system, e.g., spleen or thymus. The present invention provides methods of treating, e.g., immunogenic tumors, non-immunogenic tumors, dormant tumors, virus-induced cancers, e.g., epithelial cell cancers, endothelial cell cancers, squamous cell carcinomas, papillomavirus, adenocarcinomas, lymphomas, carcinomas, melanomas, leukemias, myelomas, sarcomas, teratocarcinomas, chemically-induced cancers, metastasis, and angiogenesis. The invention also contemplates reducing tolerance to a tumor cell or cancer cell antigen, e.g., by modulating activity of T cell infiltration and activation.

A method according to the invention of treating, delaying the progression of, preventing a relapse of, alleviating a symptom of, or otherwise ameliorating a human, mammal, or animal subject afflicted with a neoplasm can include administering a therapeutically effective amount of the compound, product and/or pharmaceutical composition, so that anti-neoplastic activity occurs. For example, the anti-neoplastic activity can be anticancer activity. For example, the anti-neoplastic activity can include slowing the volume growth of the neoplasm, stopping the volume growth of the neoplasm, or decreasing the volume of the neoplasm. The neoplasm can include a solid tumor, a malignancy, a metastatic cell, a cancer stem cell. The neoplasm can include a carcinoma, a sarcoma, an adenocarcinoma, a lymphoma, or a hematological malignancy. The neoplasm can be refractory to treatment by chemotherapy, radiotherapy, and/or hormone therapy. The compound, product and/or pharmaceutical composition can be administered to prevent relapse of the neoplasm. The compound, product and/or pharmaceutical composition can be administered as an adjuvant therapy to surgical resection. The compound, product and/or pharmaceutical composition can be administered, for example, orally and/or intravenously.

A method according to the invention also includes treating, delaying the progression of, preventing a relapse of, alleviating a symptom of, or otherwise ameliorating a disease or disorder in a human, mammal, or animal subject afflicted with that disease or disorder. In some embodiments, the disease or disorder is selected from the group consisting of an autoimmune disease, an inflammatory disease, inflammatory bowel diseases, arthritis, autoimmune demyelination disorder. Alzheimer's disease, stroke, ischemia reperfusion injury, multiple sclerosis and other neuron degenerative diseases.

Administration of the compounds, products and/or pharmaceutical compositions to a patient suffering from a disease or disorder is considered successful if any of a variety of laboratory or clinical results is achieved. For example, administration is considered successful one or more of the symptoms associated with the disease or disorder is alleviated, reduced, inhibited or does not progress to a further, i.e., worse, state. Administration is considered successful if the disorder, e.g., an autoimmune disorder, enters remission or does not progress to a further, i.e., worse, state.

In some embodiments, the compounds, products and/or pharmaceutical compositions described herein are administered in combination with any of a variety of known therapeutics, including for example, chemotherapeutic and other anti-neoplastic agents, anti-inflammatory compounds and/or immunosuppressive compounds, a cytokine or cytokine antagonist, such as IL-12, interferon-alpha, or anti-epidermal growth factor receptor. In some embodiments, the compounds, products and/or pharmaceutical compositions described herein are useful in conjunction with any of a variety of known treatments including, by way of non-limiting example, surgical treatments and methods, radiation therapy, chemotherapy and/or hormone or other endocrine-related treatment.

These “co-therapies” can be administered sequentially or concurrently. The compounds, products and/or pharmaceutical compositions described herein and the second therapy can be administered to a subject, preferably a human subject, in the same pharmaceutical composition. Alternatively, the compounds, products and/or pharmaceutical compositions described herein and the second therapy can be administered concurrently, separately or sequentially to a subject in separate pharmaceutical compositions. The compounds, products and/or pharmaceutical compositions described herein and the second therapy may be administered to a subject by the same or different routes of administration. In some embodiments, the co-therapies of the invention comprise an effective amount of the compounds, products and/or pharmaceutical compositions described herein and an effective amount of at least one other therapy (e.g., prophylactic or therapeutic agent) which has a different mechanism of action than the compounds, products and/or pharmaceutical compositions described herein. In some embodiments, the co-therapies of the present invention improve the prophylactic or therapeutic effect of the compounds, products and/or pharmaceutical compositions described herein and of the second therapy by functioning together to have an additive or synergistic effect. In certain embodiments, the co-therapies of the present invention reduce the side effects associated with the second therapy (e.g., prophylactic or therapeutic agents).

Also provided are methods of treating extramedullary hematopoiesis (EMH) of cancer, EMH is described (see, e.g., Lane, et al., 2002; Rao, et al., 2003).

The broad scope of this invention is best understood with reference to the following examples, which are not intended to limit the inventions to the specific embodiments.

### Therapeutic Compositions, Methods

Therapeutic Composition can be formulated in a pharmaceutical composition comprising a therapeutically effective amount of the Compound of the Invention and a pharmaceutical carrier. A “therapeutically effective amount” is an amount sufficient to provide the desired therapeutic result. Preferably, such amount has minimal negative side effects. The amount of Therapeutic Composition administered to treat a condition treatable with the Compound of the Invention is based on inhibiting Arf1 GTPase activity, lipolysis reporter, lipid droplet formation, autophagy, and a surrogate upstream or downstream regulator of Arf1 activity by assays known in the art. The therapeutically effective amount for a particular patient in need of such treatment can be determined by considering various factors, such as the condition treated, the overall health of the patient, method of administration, the severity of side-effects, and the like. In the tumor context, suitable activity of the Compound of the Invention would be, e.g., T cell infiltrate and activation in tumor sites, expression of inflammatory cytokines such as IL-1β and IFNγ or T cell related chemokines CCL5, CXCL-10, CXCL-11, and CCL22, or T-cell activation markers IFNγ, perforin, GzmA, GzmB, from these infiltrating cells, increased levels of DAMPs or ER stress markers or MIC-I/MHC-II or IFNγ in biological samples.

Typical veterinary, experimental, or research subjects include monkeys, dogs, cats, rat, mice, rabbits, guinea pigs, horses, and humans.

For any of the methods of treating a subject described herein, the present invention provides effective dosing ranges, dosing frequencies, and plasma concentrations of the compounds. In various embodiments, the pharmaceutical composition is administered at a dosage: (a) from about 1 mg/m² to about 5,000 mg/m² (LV.) or from about 1 mg/m² to about 50,000 mg/m² (PO); (b) from about 2 mg/m² to about 3,000 mg/m² (LV.) or from about 10 mg/m² to about 50,000 mg/m² (PO). In various embodiments, the compound of the present invention can be administered every other day (Q2D), daily (QD), or twice a day (BID). In one embodiment, the pharmaceutical composition is administered orally and no more than four times a day (QID).

In some embodiments, the disease or disorder can be treated by administering the compound, product and/or pharmaceutical composition as follows. The blood molar concentration of the compound can be at least an effective concentration and less than a harmful concentration for a first continuous time period that is at least as long as an effective time period and shorter than a harmful time period. The blood molar concentration can be less than the effective concentration after the first continuous time period. For example, the effective concentration can be about 0.1 μM, about 0.2 μM, about 0.5 μM, about 1 μM, about 2 μM, about 3 μM, about 4 μM, about 5 μM, about 6 μM, about 10 μM, or another concentration determined to be effective by one of skill in the art. For example, the harmful concentration can be about 1 μM, about 3 μM, about 10 μM, about 15 μM, about 30 μM, about 100 μM, or another concentration determined to be harmful by one of skill in the art. For example, the effective time period can be about 1 hour, 2 hours, about 4 hours, about 6 hours, about 8 hours, about 10 hours, about 12 hours, about 24 hours, or another time period determined to be effective by one of skill in the art. For example, the harmful time period can be about 12 hours, about 24 hours, about 48 hours, about 72 hours, about 144 hours, or another time period determined to be harmful by one of skill in the art.

In some embodiments, the therapeutically effective amount of the compound, product and/or pharmaceutical composition is selected to produce a blood concentration greater than the IC₅₀ of cells of the tumor and less than the IC₅₀ of normal cells. In some embodiments, the therapeutically effective amount is selected to produce a blood concentration sufficiently high to kill cells of the tumor and less than the IC₅₀ of normal cells.

In some embodiments, the compound, product and/or pharmaceutical composition is administered orally in a dosage form, for example, a tablet, pill, capsule (hard or soft), caplet, powder, granule, suspension, solution, gel, cachet, troche, lozenge, syrup, elixir, emulsion, oil-in-water emulsion, water-in-oil emulsion, and/or a draught.

In some embodiments according to the present invention, a composition for reducing or inhibiting the replication or spread of neoplastic cells includes a set of particles selected by the following method. A compound according to Formula I or a salt or solvate thereof can be provided.

In another aspect, the present invention provides a pharmaceutical composition that comprises the Compound of the Invention, and a pharmaceutically-acceptable excipient, carrier, or diluent. In one feature, the composition is suitable for oral, nasal, topical, rectal, vaginal or parenteral administration, or intravenous, subcutaneous or intramuscular injection.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the mammal being treated and the particular mode of administration. The amount of active ingredient, which can be combined with a carrier material to produce a single dosage form, will generally be that amount of the compound which produces a therapeutic effect. Generally, out of 100%, this amount will range, for example, from about 1% to about 99% of active ingredient, from about 5% to about 70%, from about 10% to about 30%.

Therapeutic compositions or formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the Compound of the Invention as an active ingredient. The Compound of the Invention may also be administered as a bolus, electuary or paste.

In solid dosage forms of the invention for oral administration (capsules, tablets, pills, dragees, powders, granules and the like), the Compound of the invention is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the Compound of the Invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxypropyl-.beta.-cyclodextrm, may be used to solubilize compounds.

Besides inert diluents, the oral compositions can also include adjuvants such as wetting agents, emulsifying and suspending agents, sweetening, flavoring, coloring, perfuming and preservative agents. Suspensions, in addition to one or more Compounds of the Invention, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more Compounds of the Invention, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention. Formulations of the present invention which are suitable for vaginal administration also include pessaries, tampons, creams, gels, pastes, foams or spray formulations containing such carriers as are known in the art to be appropriate.

Dosage forms for the topical or transdermal administration of a composition according to the invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain. In addition to the Compound of the Invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ophthalmic formulations, eye ointments, powders, solutions and the like, are also contemplated as being within the scope of this invention.

Pharmaceutical compositions of this invention suitable for parenteral administration comprise one or more compounds according to the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of the composition according to the invention, it is desirable to slow its absorption by the body from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered composition is accomplished by dissolving or suspending the compound in an oil vehicle. One strategy for depot injections includes the use of polyethylene oxide-polypropylene oxide copolymers wherein the vehicle is fluid at room temperature and solidifies at body temperature.

In an embodiment, the pharmaceutically acceptable excipient, carrier, or diluent comprises a lipid for intravenous delivery. The lipid can be: phospholipids, synthetic phophatidylcholines, natural ph sphatidylcholines, sphingomyelin, ceramides, phophatidylethanolamines, phosphatidylglycerols, phosphatide acids, cholesterol, cholesterol sulfate, and hapten and PEG conjugated lipids. The lipid may be in the form of nanoemulsion, micelles, emulsions, suspension, nano suspension, niosomes, or liposomes. In an embodiment, the pharmaceutically acceptable excipient, carrier, or diluent is in a form of micellar emulsion, suspension, or nanoparticle suspension, and it further comprises an intravenously acceptable protein, e.g., human albumin or a derivative thereof, for intravenous delivery.

In an embodiment, the pharmaceutically acceptable excipient, carrier, or diluent comprises a waxy material for oral delivery. The waxy material may be mono-, di-, or triglycerides, mono-, di-fatty acid esters of PEG. PEG conjugated vitamin E (vitamin E TPGs), and/or Gelucire. The Gelucire can be selected from Gelucire 44/14, Gelucire 43/01. Gelucire 50/02, Gelucire 50/13, Gelucire 37/02, Gelucire 33/01, Gelucire 46/07, and Gelucire 35/10. In an embodiment, the pharmaceutically acceptable excipient, carrier, or diluent is selected from capryol, transcutol hp, labrafil M, labrasol, triacetin, pharmasolv, ethanol, poly vinyl pyrrolidine, carboxymethyl cellulose, tween 20, and tween 80. In an embodiment, the pharmaceutically acceptable excipient, e.g., Gelucire 44/14, is mixed with a surfactant, which can be Tween 80 or Tween 20. These embodiments of pharmaceutical compositions can be further formulated for oral administration.

The Compound of the Invention can be synthesized using commercially available starting materials and processes well known to one skilled in the art of organic chemistry. In Examples 13-14, the present invention provides a manufacturing process for some of the claimed compounds.

According to one or more embodiments of the present invention, a small molecule Arf1 inhibitor refers to any low molecular-weight drug that shows inhibitory activity against Arf1. Compared to larger molecular weight pharmaceuticals such as proteins, peptides, and carbohydrates, small molecules can more easily penetrate cell membranes and the blood brain barrier. These molecules tend to incur lower process development and manufacturing costs.

### BRIEF DESCRIPTION OF THE DRAWING

FIG. 1, Du 101 and Du 102 are potent inhibitors of arf1 activation.

FIG. 2, in vitro cell line screening showing the effects of the arf1 inhibitor compounds 102-107, Du 102 on the colorectal cancer cell lines HT29 and LOVO cells have some tumor suppressive effect.

FIG. 3, in vitro cell line screening showed that the arf1 inhibitor compound 102-107. du102, had Tumor suppression effect on the hepatoma cell line Huh7.

FIG. 4, in vitro cell line screening shows that arf1 inhibitor compounds 102-107. Du 102 have potency tumor suppressive effect against lung cancer cells H460 and A549.

FIG. 5, in vitro cell line screening shows that arf1 inhibitor compounds 102-107. Du 102 have potency tumor suppressive effect against lung cancer cells H460 and A549.

FIG. 6, in vitro cell line screening reveals that arf1 inhibitor compounds 103-104, 107, 109. Dul02 are effective tumor suppressive effects against colorectal cancer cells cell lines HT29 and LoVo cells, liver cancer cell Huh7, lung cancer cell H460 and A549.

FIG. 7, arf1 inhibitors exert dose-dependent inhibitory effects on tumor cells.

FIG. 8, a Drosophila screen revealed that arf1 inhibitors including Du 101 and du102 had good in vivo tumor suppressive effects.

FIG. 9, the toxicity of Du 101 up to 50 mg/kg administered by oral gavage to mice for 21 consecutive days treatment.

FIG. 10, administration of Du 101 up to 50 mg/kg by oral gavage for 60 days to mice induced toxic effects.

FIG. 11, liquid phase profile of the intermediate compounds of the Du 101 synthesis scheme 1 in example 16, ADC1 refers to compound 2, Dad1 refers to compound 3, and msd1 refers to compound 4.

### EXAMPLES

### Example 1. Du 101 and Du 102 are Potent Inhibitors of Arf1 Activation

Active Arf1 was detected by using The Thermo Scientific Pierce Active Arf1 Pull-Down and Detection Kit (Cat #16121) with liver cancer Huh-7 cell lysate. It is a complete kit for selective enrichment and detection of GTP-bound Arf1 GTPase through specific protein interaction with the GGA3 protein-binding domain (FIG. 1).

### Example 2. In Vitro Cell Line Screening Shows that Arf1 Inhibitors Including Du 101 and Du 102 have Some Tumor Suppressive Effect

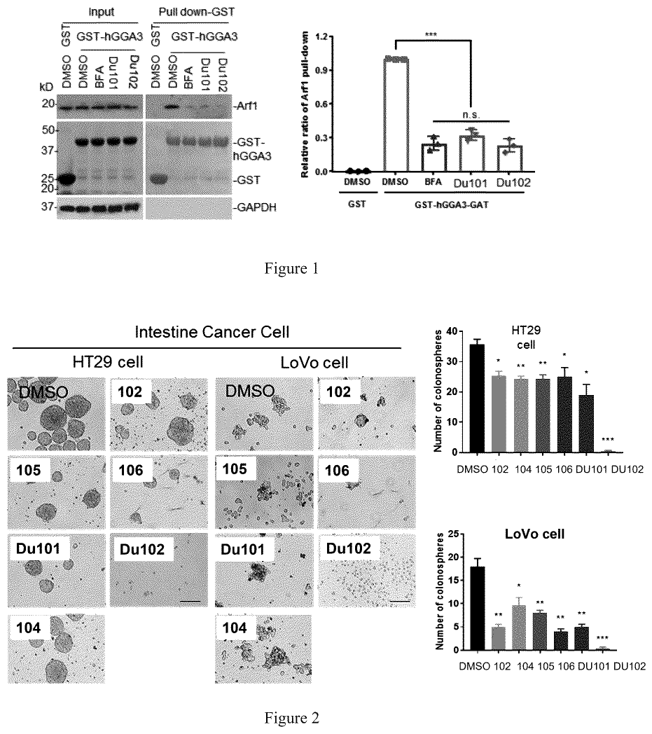

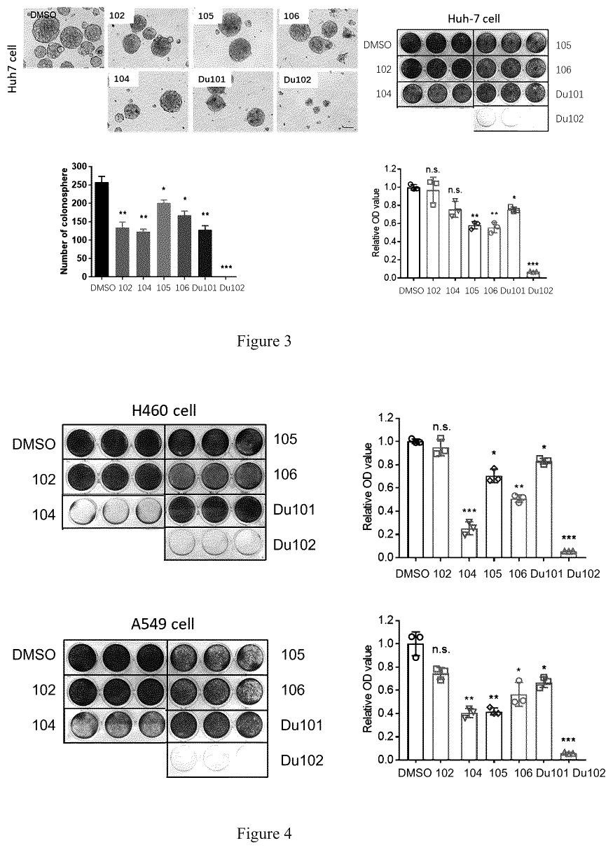

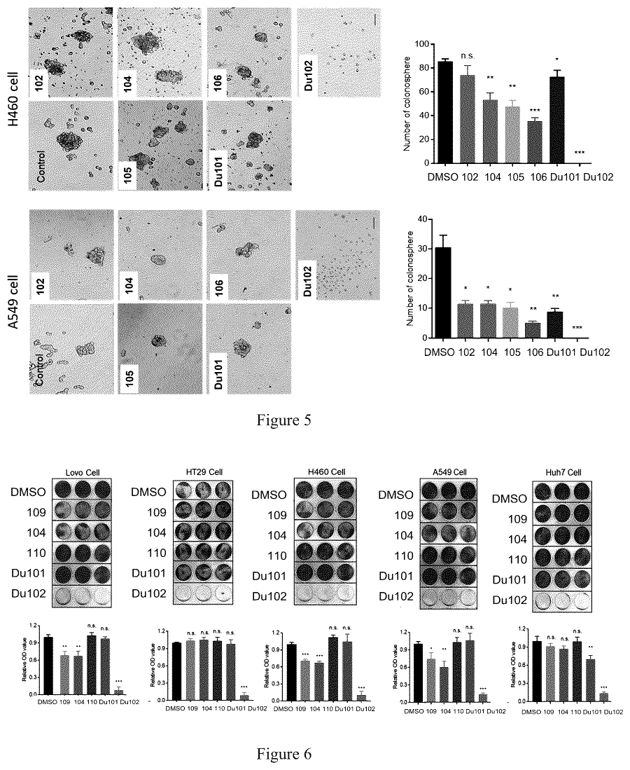

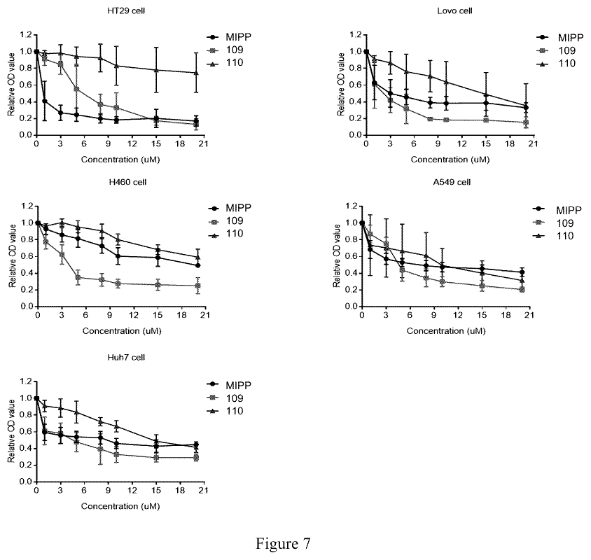

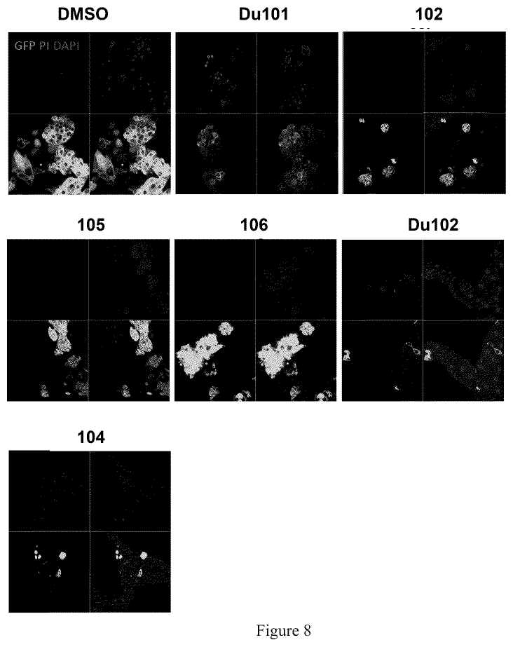

In different tumor cytotoxicity experiments in vitro (FIG. 2-FIG. 7), including colorectal cancer cell lines HT29 and LoVo cells, liver cancer cell Huh7, lung cancer cells H460 and A549, the arf1 inhibitor could all inhibit tumor cell growth (FIG. 2-FIG. 6), and there was a dose-dependent manner (FIG. 7).

### Example 3. Drosophila Screens Show that Arf1 Inhibitors Including Du 101 and Du102 have Good In Vivo Tumor Suppressive Effects

In the Drosophila gut by inducing the expression of the pro-oncogene rasvl2 GFP, which forms intestinal tumors in flies, feeding an arf1 inhibitor significantly induced cell death (PI staining) in the fly gut (FIG. 8)

### Example 4. In Vivo Druggability Experiments in Mice Show Significant Tumor Suppressive Effects of the Arf1 Inhibitors Du 101 and Du 102

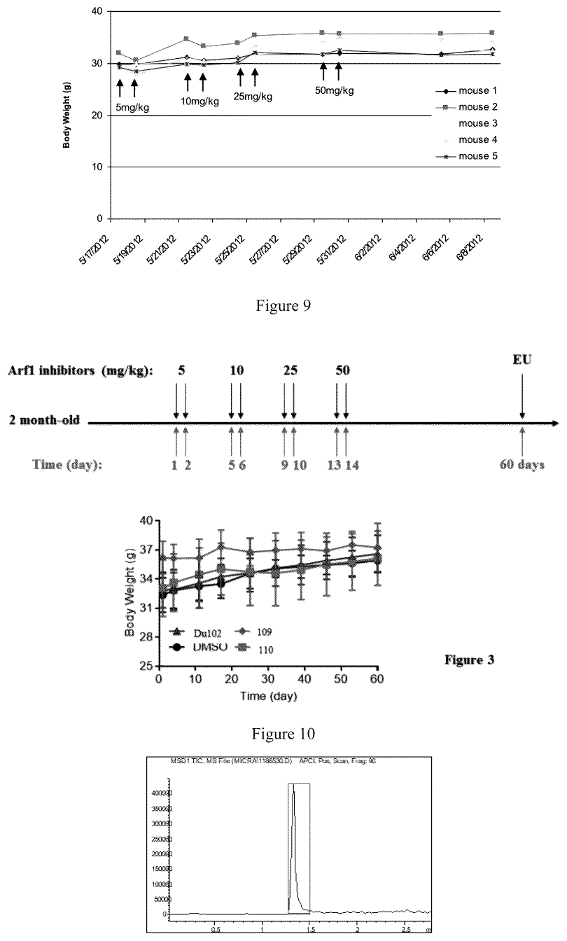

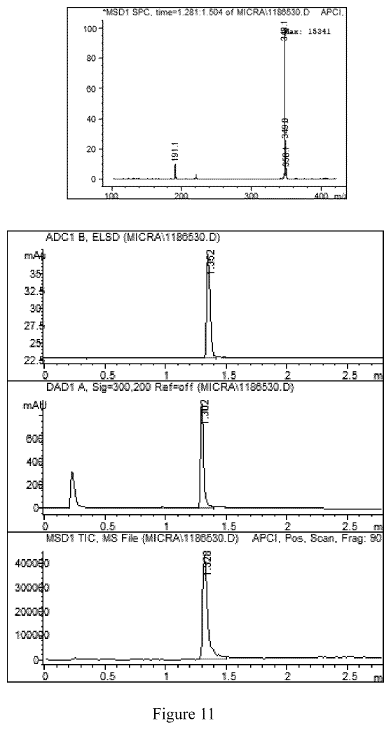

As shown in FIGS. 9-10, no toxic effects were observed in mice dosed with Du 101 up to 50 mg/kg by oral gavage for 21 consecutive days (FIG. 9) and du102 for 60 consecutive days (FIG. 10). These data indicated that compounds Du 101 and Du 102 could be used for selective anticancer activity.

### Example 5. Vaccination Made with Arf1 Suppressed Cells Protects Animals from Tumors

The immunogenicity of the reagents can be tested in mouse vaccination models (Obeid et al., 2007; Sagiv barfi et al., 2018). One flank of the mouse was injected with drug treated tumor cells, followed one week later by a second injection of syngeneic tumor cells. We tested the vaccine efficacy in CT-26 colon cancer, B16-F10 melanoma, and 4T1 breast cancer treated with DMSO or du102. We first tested the treated cells in athymic nude mice and found that inhibition of the arf1 inhibitor did not significantly affect the tumorigenic capacity of CT26, B16-F10 and 4T1 cells in immunodeficient mice (tables 2, 5, 8), indicating that the antitumor activity of arf1 knockdown is not through a direct cytotoxic effect on tumor cells.

TABLE 2
DU102 treatment had no significant effect
on CT-26 colon carcinoma in Athymic mice

| Days | 0 | 5 | 10 | 15 | 20 | 25 |
|---|---|---|---|---|---|---|
| DMSO | 0 | 0 | 21 | 170 | 480 | 1023 |
| Du102 | 0 | 0 | 15 | 117 | 417 | 859 |

TABLE 3
DU102 treatment reduces tumor size (mm [symbol] ) in
CT-2 [symbol] colon cancer model in BALB/c mice
Left side with drug treatment

| Days | 0 | 5 | 10 | 15 | 20 | 25 | 30 |
|---|---|---|---|---|---|---|---|
| DMSO | 0 | 15 | 52 | 123 | 296 | 553 | 893 |
| Du102 | 0 | 0 | 2 | 8 | 57 | 81 | 178 |

[symbol]

indicates data missing or illegible when filed

TABLE 4
Vaccination with DU102-treated CT-26 cells on left side
protects BALB/c mice from developing tumors on right side

| Days | 0 | 5 | 10 | 15 | 20 | 25 | 30 |
|---|---|---|---|---|---|---|---|
| DMSO | 0 | 0 | 9.7 | 16.3 | 23.5 | 124.7 | 211.7 |
| Du102 | 0 | 0 | 0 | 0. [symbol] | 3.7 | 7.6 | 14.1 |

[symbol]

indicates data missing or illegible when filed

TABLE 5
DU102 treatment had no significant effect
on B16-F10 melanoma in Athymic mice

| Days | 0 | 5 | 10 | 15 | 20 | 25 |
|---|---|---|---|---|---|---|
| DMSO | 0 | 6.7 | 95.4 | 242.5 | 517.7 | 1140.5 |
| Du102 | 0 | 7.3 | 51.8 | 202.7 | 355.9 | 853.4 |

TABLE 6
DU102 treatment reduces tumor size (mm [symbol] ) in
B16-F10 melanoma model in C57B/6 mice
Left side with drug treatment

| Days | 0 | 5 | 10 | 15 | 20 | 25 | 30 |
|---|---|---|---|---|---|---|---|
| DMSO | 0 | 1 [symbol] | [symbol] 1 | 132 | 297 | [symbol] 3 | 893 |
| DU102 | 0 | 0 | 2 | 8 | 57 | 81 | 177 |

[symbol]

indicates data missing or illegible when filed

TABLE 7
Vaccination with DU102-treated B16-F10 cells on left side
protects C57B/6 mice from developing tumors on right side

| Days | 0 | 5 | 10 | 15 | 20 | 25 | 30 |
|---|---|---|---|---|---|---|---|
| DMSO | 0 | 0 | 0 | 3.5 | 33.5 | 197.4 | 405.5 |
| DU102 | 0 | 0 | 0 | 0.6 | 1.3 | 9.1 | 21.3 |

TABLE 8
DU102 treatment had no significant effect
on 4T1 breast carcinoma in Athymic mice

| Days | 0 | 5 | 10 | 15 | 20 | 25 |
|---|---|---|---|---|---|---|
| DMSO | 0 | 21.3 | 41.2 | 169.7 | 402.7 | 891.5 |
| Du102 | 0 | 21.7 | 41.3 | 163.3 | 385.4 | 793.2 |

TABLE 9
DU102 treatment reduces tumor size (mm [symbol] ) in
4T1 breast carcinoma model in BALB/c mice
Left side with drug treatment

| Days | 0 | 5 | 10 | 15 | 20 | 25 | 30 |
|---|---|---|---|---|---|---|---|
| DMSO | 0 | 1.3 | 9.4 | 57.4 | 157.2 | 357.6 | 815.4 |
| DU102 | 0 | 0 | 2.3 | 18.4 | 63.5 | 102.4 | 173.6 |

[symbol]

indicates data missing or illegible when filed

TABLE 10
Vaccination with DU102-treated 4T1 cells on left side protects
BALB/c mice from developing tumors on right side

| Days | 0 | 5 | 10 | 15 | 20 | 25 | 30 |
|---|---|---|---|---|---|---|---|
| DMSO | 0 | 0 | 0 | 4.9 | 19.1 | [symbol] 1.3 | 241.7 |
| DU102 | 0 | 0 | 0 | 0 | 3.2 | 7.9 | 16.4 |

[symbol]

indicates data missing or illegible when filed

We then injected DMSO- or du102 treated CT26 cells into the left flank of BALB/c mice and re challenged the mice one week later with untreated CT-26 cells in the right flank. Tumors in mice injected with CT-26 cells treated with DMSO grew progressively on the left and right sides. Dul02 treatment resulted in almost complete regression of both left- and right-sided tumours (tables 3 and 4). Dul02 in situ vaccination was effective not only against colon cancer but also against tumors of multiple histological types. such as melanoma (B16-F10, tables 6 and 7) and breast cancer (4T1, tables 9 and 10). These results suggest that inhibition of arf1 can trigger T cell immune responses locally and then attack cancers throughout the body.

Cells will be treated with 10 μM Du 102 or DMSO treated CT26, 4T1 and B16-F10 tumor cells (5×10⁵\1×10⁵ and 5×10⁵, respectively) were subcutaneously inoculated into 6-week-old female mice. After 7 days, equal amounts of corresponding tumor cells were seeded to the right side. Tumor cell lines were mixed with Matrigel (GIBCO, cat s̆ 354234) or resuspended in PBS and injected subcutaneously or intradermally.

### Example 6. Du 102 Treatment Induces Inflammatory Cytokines in the CT-26 Colon Cancer Model in BALB/c Mice

For IL-1 β Assay: seven days after CT26 cell lysate injection. CD11c+DCs were isolated from injected BALB/c mice and co cultured with CT26 cells treated with DMSO or du102. After 2 days, IL-10 was measured in harvested cells (Table 11). For the IFN-y assay: CD11c⁺ DC and CD8⁺ T cells were isolated from injected BALB/c mice 7 days after CT26 cell lysate injection and co cultured with CT26 cells treated with DMSO or du102. After 2 days. IFN-y was measured in harvested cells (table 11).

TABLE 11
Du102 treatment induces inflammatory cytokines
in CT-26 colon cancer model in BALB/c mice

| | IL-1β | INFγ |
|---|---|---|
| DMSO | 181 | 182 |
| DU102 | 954 | 623 |

### Example 7. Dulo2 Treatment Induces IL-1 in B16-F10 Melanoma of C57B/6 Mice. (Table 12)

TABLE 12
Du102 treatment induces inflammatory cytokine
in B16-F10 melanoma model in C57B/6 mice

| | IL-1β |
|---|---|
| DMSO | 178 |
| DU102 | 952 |

### Example 8. Dul01 and Dul02 Treatments Reduce the Number of Liver Tumors in Myc-on Mice

We used the Tet system to generate transgenic mice conditionally expressing the myc pro-oncogene in hepatocytes, tet on myc/lap TTA (lt2-myc) (shachaf et al., 2004). We crossed tre-myc mice with a transgenic line, lap TTA. in which the liver activator protein (LAP) promoter drives the expression of tetracycline trans-activator protein (TTA) in hepatocytes. We confirmed this by discontinuing the feeding of doxycycline (myc-ON) activated myc transgene expression in 3-week-old mice. Subsequently. all transgenic mice overexpressing myc succumbed to liver tumors with a mean latency to tumor onset of 12 weeks, as previously reported (shachaf et al., 2004). In this lap TTA/tet-off myc conditional transgenic mouse model, myc overexpression in adult mice reproducibly induces liver cancer, similar to hepatocellular carcinoma and/or hepatoblastoma. The transgenic tumor invades the entire liver locally and is frequently associated with malignant peritoneal effusions that spread to the thoracic cavity by metastasis and invade the lung parenchyma.

For the mouse liver tumor model, mating cages and weaning cages were administrated by a grain based rodent diet (Bio-Serv. Cat #14-727-450) containing 200 mg/kg doxycycline, and the mice were induced tumors at 6 weeks of age, by changing to a normal diet. Mice were divided into three groups, control, Du 101 treated and du102 treated. The control group was given 100 by gavage using a feeding tube 100 μl/10 g body weight (BW) 20% DMSO+80% corn oil. Du 101 treated groups were treated with 25 mg/ml Du 101. The stock solution was diluted 1:4 with corn oil to make the working solution. Dul02 treatment groups were treated with 25 mg/ml du102 stock solution diluted 1:4 with corn oil to make working solutions. The mixed working solution was administered to mice by gavage using a feeding tube. Starting from the second week of myc-0n, 5 injections per week (starting Monday and Friday) were given for 4 weeks, mice were monitored every 2 days, and 10 weeks later mice were euthanized and analyzed for tumor growth. Table 13 indicates that Du 101 and Du 102 can reduce myc induced liver tumors that are overexpressed in mice.

TABLE 13
DU101 and Du102 treatment reduces
liver tumor numbers in MYC-ON mice

| | |
|---|---|
| DMSO | 105.3 |
| DU101 | 44.7 |
| DU102 | 26.7 |

### Example 9. Du 102 Treatment Induces the Expression of Chemokines and T Cell Activation Markers in Myc-on Mice

Chemokines and T cell activation markers were measured by quantitative RT-PCR in DMSO or du102 treated MYC-ON mice. CD8 T cell infiltration and immunostimulatory cytokine expression were increased as measured by quantitative RT-PCR. Compared with mice treated with the MSO control, du102 treatment significantly increased the expression of many T cell associated chemokines CCL5. cxcl-10. cxcl-11 and CCL22 in myc-on mice (table 14). CCL5 and CXCL10 chemokines stimulate CD4+ and CD8+ lymphocytes tumor infiltration (Parkes et al., 2017), and CXCL10 and CXCL11 are T cell associated chemokines. Furthermore, real-time PCR revealed increased expression of the T cell activation markers ifny, perforin, gzma, gzmB, and IL-1 β in mice treated with Du 102 compared with mice treated with DMSO (table 14). Our real-time PCR also showed that the immune checkpoint inhibitor PD-L1 expression was significantly decreased in mice treated with du102 compared with mice treated with DMSO control in myc-on mice (table 14). Collectively, these data indicated that du102 treatment triggered T cell infiltration and activation, leading to liver tumor cell death and prolonged the survival time of myc-on mice.

TABLE 14
DU102 treatment induces chemokines and T-cell activation in MYC-ON mice

| | Ccl5 | Cxcl10 | Cxcl11 | Col22 | GzmA | GzmB | Perforin | IL-1β | INFγ | PD-L1 |
|---|---|---|---|---|---|---|---|---|---|---|
| DMSO | 1.0 | 1.1 | 1.0 | 1.0 | 1.0 | 1.0 | 1.0 | 1.0 | 1.1 | 1.0 |
| DU102 | 2.4 | 3.1 | 2.8 | 2.5 | 2.3 | 1.9 | 2.2 | 2.5 | 3.2 | 0.4 |

### Example 10. Du 102 Treatment Induces the Expression of T Cell Activation Markers of 4T1 Breast Cancer in BALB/c Mice

TABLE 15
DU102 treatment induces T-cell activation
of 4T1 breast carcinoma in BALB/c mice

| | GzmA | Gzm8 | Perforin | IL-1β | INFγ | PD-L1 |
|---|---|---|---|---|---|---|
| DMSO | 1.0 | 1.0 | 1.0 | 1.0 | 1.0 | 1.0 |
| DU102 | 2.2 | 2.3 | 2.5 | 2.2 | 2.1 | 0.5 |

### Example 11. Du 102 Treatment Induces MHC Expression and T Cell Infiltration in Myc-on Mice

Du 102 treatment increases MHC expression. CD4 and CD8 T cell infiltration as quantified by Q-PCR in myc-on mice (Table 16).

TABLE 16
DU102 treatment induces MHC expression
and T-cell infiltration in MYC-ON mice

| | MHC-1 | MHC-II | CD4 | CD8 |
|---|---|---|---|---|
| DMSO | 0.6 | 0.7 | 1.0 | 2.0 |
| DU102 | 51.7 | 43.6 | 13.2 | 13.5 |

MHC-1 or MHC-II positive infiltrating CD4+ and CD8+ T-cells measured by immunohistochemical staining of representative section.

### Example 12. Du 102 Treatment Reduces Lung Metastasis of B16-F10 Melanoma in C57B/6 Mice

B16-f10 tumor cells (treated with DMSO or 10 μM Du 102 treatment) was transferred intravenously via tail vein injection. Lungs were removed 15 days after injection and fixed in fekete's solution overnight. Three investigators in a blinded manner statistically available (Table 17).

TABLE 17
Du102 treatment reduces lung metastases of B16-F10
melanoma in C57B/6 mice (surface tumor number)

| | |
|---|---|
| DMSO | 39 |
| DU102 | 5 |

### Example 13 Antitumor Effect of Du 102 in Myc-on Mice is Neutralized by CD4 or CD8 Antibodies

The antitumor effect of Du 102 was neutralized by CD4 or CD8 antibodies in myc-on mice (table 18). Cell depletion was verified using FACS and IHC analyses.

TABLE 18
The anti-tumor effects of DU102 are neutralized by
anti- CD4 or/anti-CD8 antibodies in MYC-ON mice

| DMSO + | Du102 + | DMSO + | Du102 + | Du102 + |
|---|---|---|---|---|
| Isotype | Isotype | Anti-CD4 | Anti-CD4 | Anti-CD4/CD8 |
| 91.3 | 27.5 | 51.8 | 69.5 | 89.7 |

For the myc-on liver tumor model, antibodies were i.p. Mg/kg) was injected once a week for 5 consecutive weeks, the first Doxycycline was discontinued weekly after injection. Antibodies used to treat mice were as follows: anti-CD4 (clone GK1.5, BioXcell, Cat #BE0003-1), anti-CD8 a (clone 2.43, BioXcell, Cat #BE0061) Or 100 ng/mouse rat IgG (bioxcell, cat #be0094) isotype control antibody. From the second week of myc-on initiation lasted for 4 weeks, Du 102 and DMSO were administered by gavage needle as described above, mice were monitored every 2 days, and 10 weeks later mice were euthanized and analyzed for tumor growth.

### Example 14. Synergistic Antitumor Effect of Du 102 with Anti-PD-1 Antibody in Myc-on Mice

Du102 was administered in combination with anti-PD-1. We tested arf1 inhibition and PD-1 blockade in myc-on mice, PD-1 blockade was found to further reduce the number of tumors in Du 102 treated myc-on mice (Table 19).

TABLE 19
Synergistic anti-tumor effects of DU102
with anti-PD-1 antibodies in MYC-ON mice

| DMSO + | DMSO + | Du102 + | Du102 + |
|---|---|---|---|
| Isotype | Anti-PD-1 | Isotype | Anti-PD-1 |
| 92.5 | 65.3 | 42.3 | 4.4 |

For the myc-on liver tumor model, antibodies were i.p. Injection once a week for 5 weeks, and doxycycline was discontinued weekly after the first injection. Antibodies used to treat mice were as follows: 100 ug/mouse Armenian hamster anti mouse PD1 (J43, bioxcell, cat #be0033-2) and 100 ug/mouse Armenian hamster isotype IgG (bioxcell. cat #bp0091) was used as controls. For 4 weeks from the second week of myc-on, Du 102 and DMSO were administered by gavage needle as described above, mice were monitored every 2 days. and 10 weeks later mice were euthanized and analyzed for tumor growth. Coadministration of anti-PD-1 antibodies may allow the use of lower, less toxic doses of du102, thus avoiding known side effects.

### Example 15. Therapeutic Effects of Du 102. Du 101 in Breast Cancer, Head and Neck Cancer, Lung Cancer, Ovarian Cancer, Pancreatic Cancer, Colorectal Cancer, Prostate Cancer, Renal Cell Carcinoma, Melanoma, Hepatocellular Carcinoma, Cervical Cancer, Sarcoma, Brain Tumor, Gastric Cancer, Multiple Myeloma, Leukemia, Lymphoma

Humanized tumor models were constructed by inoculating human breast, head and neck, lung, ovarian, pancreatic, colorectal, prostate, renal cell, melanoma, hepatocellular, cervical, sarcoma. brain, gastric, multiple myeloma, leukemia, lymphoma tumor tissues or cell lines into humanized mice subcutaneously. DMSO or Du 102 were administered by gavage Du 101 processing. Immunostimulatory cytokine expression was detected, as measured by quantitative RT-PCR. Detection of T cell activation markers ifny, perforin, gzma, gzmB, and IL-1 β in mice by FACS expression. And the therapeutic effects of du102, Du 101 treatment on breast cancer, head and neck cancer, lung cancer, ovarian cancer, pancreatic cancer, colorectal cancer, prostate cancer, renal cell carcinoma, melanoma, hepatocellular carcinoma, cervical cancer, sarcoma, brain tumor, gastric cancer, multiple myeloma, leukemia, lymphoma were examined by measuring tumor growth, collecting mice survival data. Or by combining mouse derived breast, head and neck, lung. ovarian, pancreatic, colorectal, prostate, renal cell carcinoma, melanoma, hepatocellular carcinoma, cervical carcinoma, sarcoma, brain tumor, gastric cancer, multiple myeloma, leukemia, lymphoma tumor tissues or cell lines were inoculated subcutaneously in wild-type mice to construct tumor models. DMSO- or du102-. Du 101- treatments were administered by gavage. Immunostimulatory cytokine expression was detected, as measured by quantitative RT-PCR. Detection of T cell activation markers ifny, perforin, gzma, gzmB, and IL-1 β in mice by FACS expression. And the therapeutic effects of du102-, Du 101- treatment on breast cancer, head and neck cancer, lung cancer, ovarian cancer, pancreatic cancer, colorectal cancer, prostate cancer, renal cell carcinoma, melanoma, hepatocellular carcinoma, cervical cancer, sarcoma, brain tumor, gastric cancer, multiple myeloma, leukemia, lymphoma were examined by measuring tumor growth, collecting mice survival data. Or mouse models of primary breast, head and neck, lung. ovarian, pancreatic, colorectal, prostate, renal cell carcinoma, melanoma, hepatocellular, cervical, sarcoma, brain tumor, gastric cancer, multiple myeloma, leukemia, lymphoma resulting from conditional knockout or knockin of the gene. DMSO or du102, Du 101 treatments were administered by gavage. Immunostimulatory cytokine expression was detected, as measured by quantitative RT-PCR. The expression of T cell activation markers ifny, perforin, gzma, gzmB, and IL-1β in mice was examined by FACS. And the therapeutic effects of du102-, Du 101- treatment on breast cancer, head and neck cancer, lung cancer, ovarian cancer, pancreatic cancer, colorectal cancer, prostate cancer, renal cell carcinoma, melanoma, hepatocellular carcinoma, cervical cancer, sarcoma, brain tumor, gastric cancer, multiple myeloma, leukemia, lymphoma were examined by measuring tumor growth, collecting mice survival data.

### Example 16. Therapeutic Effects of Du102, Du 101 in Autoimmune Disease Inflammatory Disease Inflammatory Bowel Disease Arthritis Autoimmune Demyelinating Disease

DMSO or du102, du101 treatment was administered by gavage or intracranial injection in autoimmune, inflammatory bowel, arthritis, autoimmune demyelinating mouse disease models. Immunostimulatory cytokine expression was detected, as measured by quantitative RT-PCR. Detection of T cell activation markers ifny, perforin, gzma, gzmB, and IL-1β in mice by FACS expression. And by sectioning. the mice incidence was detected.

### Example 17. Preparation of Compound 1H-Indole-5-Carbaldehyde 6,7,8,9-Tetrahydro-5H-cyclohepta[4,5]thieno[2,3-d]pyrimidin-4-ylhydrazone, Scheme 1

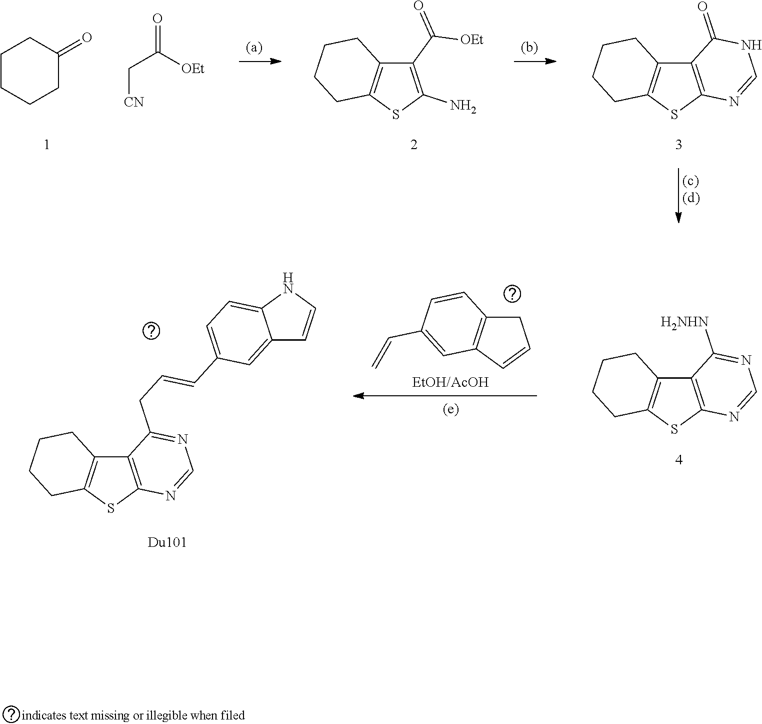

### Synthesis of Du 101

Compounds referred below are shown in the schematic in Scheme 1. Compounds were dissolved in DMSO and stored at −20° C.

### Compound 2

To a solution of cyclohexanone (10 mmol) in ethanol (10 mL) were added sulfur (320 mg, 10 mmol), ethyl cyanoacetate (1.07 mL, 10 mmol) and morpholine (875 μL, 40 mmol). The reaction mixture was stirred at 60° C. for 5 h. Eight hundred milligrams of 2 was obtained after purification by chromatography using dichloromethane. (Note: The starting materials can be proportionally increased to obtain more compound 2).

### Compound 3

Compound 2 was heated at 150° C. in formamide for 5 h. Upon cooling overnight, the product crystallized as slightly brownish crystals. The resulting crystals were collected and washed with a mixture of cold ethanol/water (1/1) to give the corresponding thienopyrimidone ring 3 in quantitative yield.

### Compound 4

Six hundred and fifty milligrams of 3 was dissolved in hot DMF (dimethylformamide) and ice-cooled prior to the addition of 2 equivalent of POCl₃. Upon stirring overnight, the product precipitated out. The white powder was collected and washed with cold water. Further addition of cold water into the mother liquor gave additional precipitate which is used straight away in the next step.

To a solution of chloride dissolved in methanol was added 10 equivalent of hydrazine monohydrate. The mixture was stirred for 2 h and water was added. The resulting precipitate was filtered off and washed with cold water to give the product 4.

### Du 101

To a solution of 1 equivalent of 4 in methanol was added 1.2 equivalent of Indole-5-carboxaldehyde. The mixture was stirred for 2 hours, and the resulting precipitate was collected and recrystallised from methanol to afford the Du 101. H NMR (300 MHz, DMSO) δ: 1.79 (m, 4H, 2 CH2), 2.74 (m, 2H, CH2), 3.00 (m, 2H, CH2), 6.49 (m, 1H, CH), 7.39 (m, 1H, CH), 7.42 (d, J=8.6 Hz, 1H, CH), 7.76 (s, 1H, CH), 7.84 (d, J=8.6 Hz, 1H, CH), 8.02 (s, 1H, CH), 8.45 (s, 1H, CH), 11.29 (brs, 1H, NH), 11.73 (brs, 1H, NH), 13C NMR (75 MHz, DMSO) δ: 22.0 (CH2), 22.4 (CH2), 24.6 (CH2), 26.5 (CH2), 101.8 (CH), 111.5 (CH), 118.9 (C), 120.6 (CH), 121.3 (CH), 126.2 (CH), 126.6 (C), 127.6 (C), 130.8 (C), 131.8 (C), 136.9 (C), 144.0 (CH), 148.0 (C), 154.8 (CH), 156.6 (C). ES-MS m/z 348.1 (MH+). HRMS 348.1277, found 348.1290. Anal. (C19H17N5S) C, H, N, S.

SOURCE-01

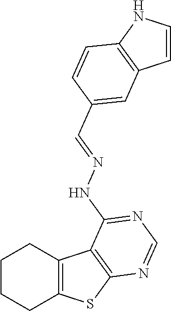 [SMILES: C(=N\Nc1ncnc2sc3c(c12)CCCC3)/c1ccc2[nH]ccc2c1]

| | | | |
|---|---|---|---|
| ID | 118653005 | 347.448 | C₁₉H₁₇N₅S |

SOURCE-01

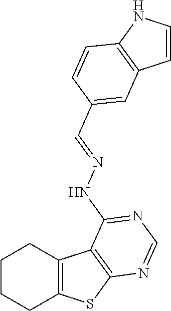 [SMILES: C(=N\Nc1ncnc2sc3c(c12)CCCC3)/c1ccc2[nH]ccc2c1]

| | | | |
|---|---|---|---|
| ID | 118653005 | 347.4448 | C₁₉H₁₇N₅S |

Data File D:\DATA\MICRA\1186530.D
Sample Name: 118653005
Instrument 1 Apr. 10, 2017 14:20:33 N6
Column: Onyx Monolithic C18 50×4.6 mm|3.75 ml/min|Columns Reg Valve
Gradient: “A”->@2.2 min->“B” (Hold 0.4 min)->@0.2 min->“A”->PostRun
PMP1, Solvent A: 0.1% TFA in Acn/H2O (2.5:97.5)
PMP1, Solvent B: 0.1% TFA in AcN
PMP1, Solvent C: 0.1% FA in Acn/H2O (2.5:97.5)
PMP1, Solvent D: 0.1% FA in AcN
Ionization mode APCI Positive

Signal 1: ADC1 B, ELSD

| Peak | RetTime Type | Width | Area | Height | Area |
|---|---|---|---|---|---|
| # | [min] | [min] | [mAu*s] | [mAu] | % |
| 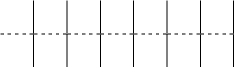 | | | | | |
| 1 | 1.352 PB | 0.0281 | 28.29734 | 14.96186 | 100.0000 |
| Totals: | | | 28.29734 | 14.96186 | |

Signal 2: DAD1 A, Sig = 300,200 Ref = off

| Peak | RetTime Type | Width | Area | Height | Area |
|---|---|---|---|---|---|
| # | [min] | [min] | [mAU*s] | [mAU] | % |
| 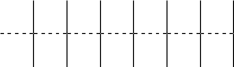 | | | | | |
| 1 | 1.302 BB | 0.0294 | 1616.86841 | 882.75543 | 100.0000 |
| Totals: | | | 1616.86841 | 882.75543 | |

Signal 3: MSD1 TIC, MS File

| Peak | RetTime Type | Width | | | Area |
|---|---|---|---|---|---|
| # | [min] | [min] | Area | Height | % |
| 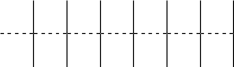 | | | | | |
| 1 | 1.328 BB | 0.0472 | 1.33299e6 | 4.31713e5 | 100.0000 |
| Totals: | | | 1.33299e6 | 4.31713e5 | |

### Example 18. Preparation of Compound 1H-indole-5-carbaldehyde 5,6,7,8,9,10-hexahydrocycloocta [4,5] thieno[2,3-d] pyrimidin-4-ylhydrazone Scheme 2

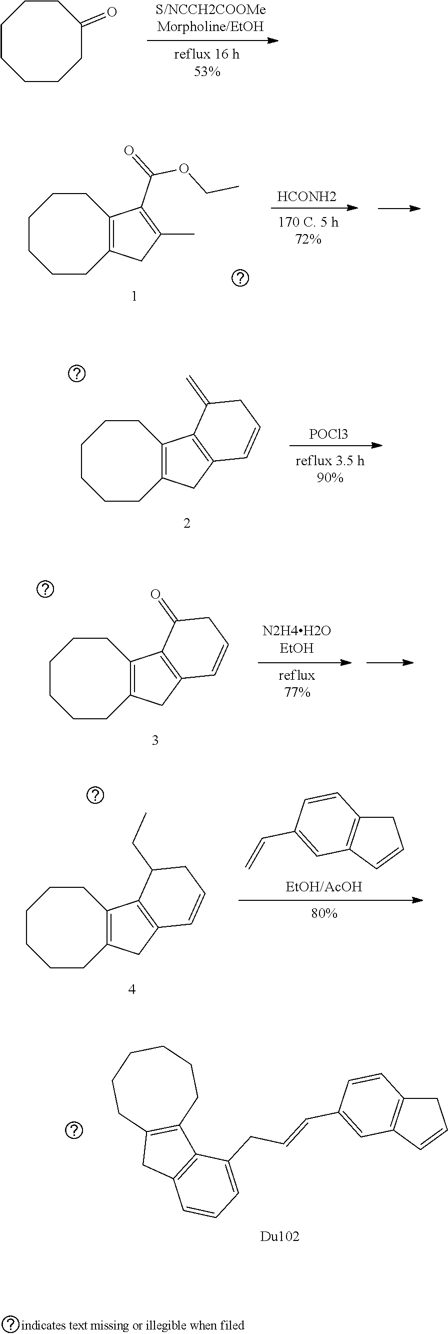

### Synthesis of Du 102

Compounds referred below are shown in the schematic in Scheme 2. Compounds were dissolved in DMSO and stored at −20° C.

### Compound 1

To a solution of cyclooctanone (10 mmol) in ethanol (10 mL) were added sulfur (320 mg, 10 mmol), ethyl cyanoacetate (1.07 mL, 10 mmol) and morpholine (875 μL, 40 mmol). The reaction mixture was stirred at 60° C. for 5 h. Eight hundred and fifty-five milligrams of 1 was obtained (yield: 34%) after purification by chromatography using dichloromethane. (Note: The starting materials can be proportionally increased to obtain more compound 1).

¹H NMR (400 MHz, CDCl₃) δ: 1.28 (m, 5H, CH₃+CH₂), 1.39 (m, 2H, CH₂), 1.50 (m, 2, CH₂), 1.56 (m, 2H, CH₂), 2.54 (m, 2H, CH₂), 2.75 (m, 2H, CH₂), 4.21 (q, 2H, J=7.5 Hz, CH₂), 5.84 (brs, 2H, NH₂). ¹³C NMR (100 MHz, CDCl₃) δ: 14.5 (CH₃), 24.6 (CH₂), 25.0 (CH₂), 25.9 (CH₂), 26.1 (CH₂), 29.2 (CH₂), 31.5 (CH₂), 58.8 (CH₂), 105.6 (C), 119.5 (C), 134.3 (C), 160.8 (C), 164.2 (C). ES-MS m/z 254.1 (MH⁺).

### Compound 2

Compound 1 was heated at 150° C. in formamide for 5 h. Upon cooling overnight, the product crystallized as slightly brownish crystals. The resulting crystals were collected and washed with a mixture of cold ethanol/water (1/1) to give the corresponding thienopyrimidone ring 2 in quantitative yield.

¹H NMR (400 MHz, DMSO) δ: 1.27 (m, 2H, CH₂), 1.42 (m, 2H, CH₂), 1.62 (m, 4H, 2 CH₂), 2.87 (m, 2H, CH₂), 3.06 (m, 2H, CH₂), 8.01 (s, 1H, CH), 12.28 (brs, 1H, NH). ¹³C NMR (100 MHz, DMSO) δ: 24.4 (CH₂), 25.3 (CH₂), 25.4 (CH₂), 26.0 (CH₂), 29.9 (CH₂), 31.5 (CH₂), 133.7 (C), 135.0 (C), 144.6 (C), 147.8 (C), 150.0 (CH), 157.7 (C). ES-MS m/z 235.1 (MH⁺).

### Compound 3

Six hundred and fifty milligrams of 2 was dissolved in hot DMF (dimethylformamide) and ice-cooled prior to the addition of 2 equivalent of POCl₃. Upon stirring overnight, the product precipitated out. The white powder was collected and washed with cold water. Further addition of cold water into the mother liquor gave additional precipitate which is used straight away in the next step.

¹H NMR (400 MHz, CDCl₃) δ: 1.25 (m, 2H, CH₂), 1.46 (m, 2H, CH₂), 1.70 (m, 4H, 2 CH₂), 2.92 (m, 2H, CH₂), 3.12 (m, 2H, CH₂), 8.67 (s, 1H, CH). ¹³C NMR (100 MHz, CDCl₃) δ: 25.0 (CH₂), 25.4 (CH₂), 26.3 (CH₂), 28.2 (CH₂), 30.3 (CH₂), 31.6 (CH₂), 128.6 (C), 129.6 (C), 142.7 (C), 151.3 (CH), 156.4 (C), 158.7 (C). ES-MS (electrospray mass spectrometry) m/z 252.1 (MH⁺, ³⁵Cl), 254.1 (MH⁺, ³⁷Cl).

### Compound 4

To a solution of chloride dissolved in methanol was added 10 equivalent of hydrazine monohydrate. The mixture was stirred for 2 h and water was added. The resulting precipitate was filtered off and washed with cold water.

¹H NMR (400 MHz, CDC₃) δ: 1.27 (m, 2H, CH₂), 1.44 (m, 2H, CH₂), 1.65 (m, 4H, 2 CH₂), 2.50 (brs, 2H, NH₂), 2.83 (m, 4H, 2 CH₂), 6.54 (brs, 1H, NH), 8.41 (s, 1H, CH). ¹³C NMR (100 MHz, CDCl₃) δ: 25.3 (CH₂), 26.0 (CH₂), 26.1 (CH₂), 27.7 (CH₂), 30.1 (CH₂), 31.6 (CH₂), 115.7 (C), 127.7 (C), 137.3 (C), 152.3 (CH), 158.7 (C), 164.8 (C). ES-MS m/z 249.0 (MH⁺), 271.0 (MNa⁺).

### Du 102

To a solution of 1 equivalent of 4 in methanol was added 1.2 equivalent of Indole-5-carboxaldehyde. The mixture was stirred for 2 hours, and the resulting precipitate was collected and recrystallised from methanol to afford the Du 102.

¹H NMR (400 MHz, DMSO) δ: 1.25 (m, 2H, CH₂), 1.44 (m, 2H, CH₂), 1.60 (m, 2H, CH₂), 1.69 (m, 2H, CH₂), 2.83 (brt, 2H, J=5.5 Hz, CH₂), 3.20 (brt, 2H, J=6.0 Hz, CH₂), 6.49 (m, 1H, CH), 7.39 (m, 1H, CH), 7.44 (d, 1H, J=8.6 Hz, CH), 7.80 (brs, 1H, CH), 7.85 (brd, 1H, J=8.6 Hz, CH), 8.03 (s, 1H, CH), 8.46 (s, 1H, CH), 11.28 (brs, 1H, NH) 11.71 (brs, 1H, NH). LC/MS, API-ES m/z 376.0 (MH⁺).

All citations herein are incorporated herein by reference to the same extent as if each individual publication or patent application was specifically and individually indicated to be incorporated by reference.

Modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

1. A method of killing a progenitor or a stem cell or a cancer stem cell or a cancer cell and inducing anti-tumor immune response, the method comprising inhibiting at least some Arf1, particularly COPI/Arf1-lipolysis-β-oxidation pathway activity in a cancer stem cell through a Arf1 pathway inhibitor, wherein the Arf1 pathway inhibitor is a compound related to formula (I): 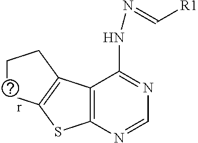
where r=1-6; R1 is selected from the group consisting of one or more substituents or unsubstituted phenyl, 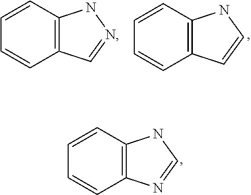
substituents selected from the group consisting of halo, C₁₋₆ alkyl, haloalkyl, OH, OCH₃, O(CH₂)nCH₃ Cyclopropyloxy group, OC(CH₃)₃, OCH(CH₃)₂, NH₂, NO₂, N (CH₃)₂, NH (CH₂)nCH₃, CN, N₃, which the median N was 1-9.

2. The method of claim 1, wherein an inhibitor targeting the arf1 pathway is a compound as follows:
TABLE 1

| | |
|---|---|
| 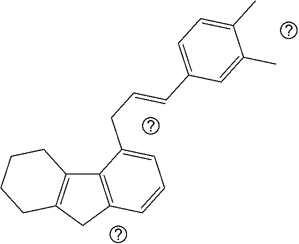 [SMILES: Cc1ccc(/C=C/Cc2cccc3c2C2=C(CCCC2)C3)cc1C] | 102 |
| 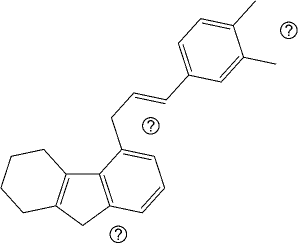 [SMILES: Cc1ccc(/C=C/Cc2cccc3c2C2=C(CCCC2)C3)cc1C] | 104 |
| 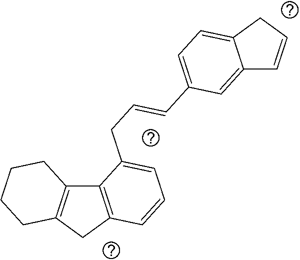 [SMILES: C1=Cc2cc(/C=C/Cc3cccc4c3C3=C(CCCC3)C4)ccc2C1] | 105 |
| 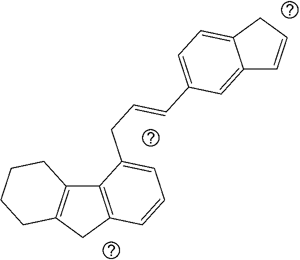 [SMILES: C1=Cc2cc(/C=C/Cc3cccc4c3C3=C(CCCC3)C4)ccc2C1] | 106 |
| 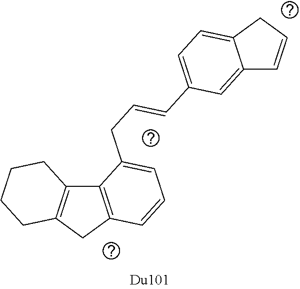 [SMILES: C1=Cc2cc(/C=C/Cc3cccc4c3C3=C(CCCC3)C4)ccc2C1] | 107 |
| 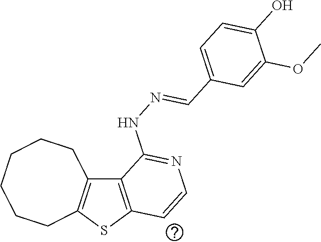 [SMILES: COc1cc(/C=N/Nc2nccc3sc4c(c23)CCCCCC4)ccc1O] | 108 |
| 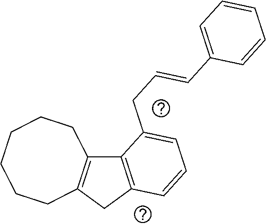 [SMILES: C(=C\c1ccccc1)/Cc1cccc2c1C1=C(CCCCCC1)C2] | 109 |
| 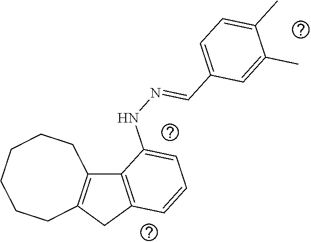 [SMILES: Cc1ccc(/C=N/Nc2cccc3c2C2=C(CCCCCC2)C3)cc1C] | 110 |
| 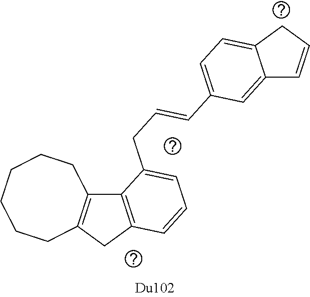 [SMILES: C1=Cc2cc(/C=C/Cc3cccc4c3C3=C(CCCCCC3)C4)ccc2C1] | 111 |

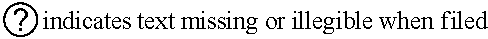

3. A method of inhibiting cellular Arf1 pathway activity in a cell,
comprising administering to the cell an effective amount of a compound selected from the compounds mentioned in claim 1, and a salt or solvate thereof, such that at least Arf1 pathway activity in the cell is reduced.

4. The method of claim 3 wherein the cell is a progenitor cell or a stem cell or a cancer stem cell.

5. The method of claim 3 wherein the cell is a cancer cell.

6. The method of claim 3 wherein cell death is induced in the cell.

7. The method of claim 3 wherein the method is carried out in vitro.

8. The method of claim 3 wherein the method is carried out in vivo.

9. A method of treating or preventing a disorder associated with Arf1 pathway activity in a subject, the method comprising administering to the subject a therapeutically effective amount of a pharmaceutical composition comprising a compound selected from claim 1, and a pharmaceutically acceptable salt or solvate thereof, such that at least Arf1 pathway activity is reduced.

10. The method of claim 9, wherein aberrant Arf1 pathway activity can be identified by assaying Arf1 GTPase activity, lipolysis reporter, lipid droplet formation, autophagy, and a surrogate upstream or downstream regulator of Arf1 activity or functions.

11. An effective amount of the compound of Formula (I) of claim 1, for use in treating a subject suffering from a cancer or tumor.

12. The compound for use of claim 11, wherein the diseases are among autoimmune diseases, inflammatory diseases, inflammatory bowel diseases, arthritis, autoimmune demyelinating diseases, Alzheimer's disease, amyotrophic lateral sclerosis (ALS), stroke, ischemia-reperfusion injury, multiple sclerosis, other neuronal degenerative diseases.

13. The compound for use of claim 11, wherein the compound inhibits growth of the cancer or tumor.

14. The compound for use of claim 11, wherein the compound reduces the size of the tumor or cancer.

15. The compound for use of claim 11, wherein the compound increases expression of MHC-I and MHC-II, and the compound increases infiltration and activation of T cells into the tumor when compared to DMSO and increases expression of T cell activation markers, such as GzmA, GzmB and Perforin.

16. The compound for use of claim 11, wherein the compound increases the expression of at least one inflammatory cytokine or chemokine, which can be selected from the group consisting of IFNγ, IL-1β, Ccl5, Cxc10, Cxc11, and Ccl22.

17. The compound for use of claim 11, wherein the compound is co-administered with at least one anti-PD-1 antibody, the compound and PD-1 blockage had a synergistic effect.

18. The compound for use of claim 11, wherein vaccination with the compound treated cancer cells protects animals from developing tumors.

19. The compound for use of claim 11, wherein the compound reduces metastasis of a cancer or tumor.

20. The compound of claim 11, wherein the cancer is selected from the group consisting of breast cancer, head and neck cancer, lung cancer, ovarian cancer, pancreatic cancer, colorectal carcinoma, prostate cancer, renal cell carcinoma, melanoma, hepatocellular carcinomas, cervical cancer, sarcomas, brain tumors, gastric cancers, multiple myeloma, leukemia, and lymphomas.

21. The method of claim 9 wherein the subject is a mammal.

22. A compound or pharmaceutically acceptable salt, solvate, or prodrug thereof, stereoisomeric are used as claimed in claim 1 in the preparation of drugs for the treatment of subjects with cancer or tumors.

23. The compound according to claim 22, wherein the cancer is breast cancer, head and neck cancer, lung cancer, ovarian cancer, pancreatic adenocarcinoma, colorectal, prostate, renal cell carcinoma, melanoma, hepatocellular carcinoma, cervical cancer, sarcoma, brain tumor, gastric cancer, multiple myeloma, leukemia, lymphoma.

24. The compound according to claim 22, wherein the compound is co administered with anti-PD-1 antibody, the compound and PD-1 blockade have synergistic effects.

25. The compound according to claim 22, wherein the compound as claimed not only kills tumor stem cells but also causes tumors specific immune responses.

26. The compound according to claim 22, wherein the compound can reduce the metastasis of cancer or tumor.

27. A kit containing at least one reagent for the diagnosis of diseases associated with arf1 pathway activity with compounds or pharmaceutically acceptable salt, solvate or prodrug thereof, stereoisomer described in claim 1.

28. The kit according to claim 27 mentioned kit, wherein at least one agent tests at least one biomarker indicating disease.

29. The kit according to claim 27, wherein at least one reagent is used to detect arf1 GTPase activity, an upstream or downstream regulator of lipolytic reporters, lipid droplet formation, autophagy, arf1 activity, or function.

30. A process for the preparation of compound 107 as claimed in claim 2 comprising:

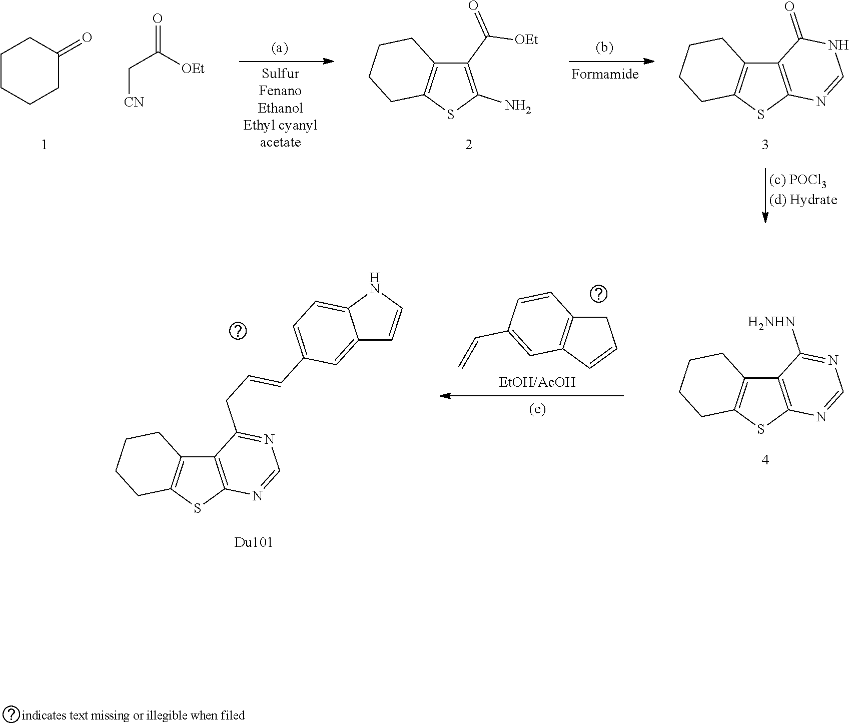

31. A process of preparing the compound of 111 in claim 2 comprising: [chemical structure]
